(19) United States
(12) Patent Application Publication
(10) Pub. No.: US 2007/0218553 A1
(43) Pub. Date: Sep. 20, 2007

(54) CELL SEPARATION APPARATUS

(76) Inventors: Horacio L. RODRIGUEZ RILO, Cincinnati, OH (US); Arthur J. Helmicki, Cincinnati, OH (US); William R. Heineman, Cincinnati, OH (US); H. Brian Halsall, Cleves, OH (US); Kevin Schlueter, St. Bernard, OH (US); Anne Maghasi, Racine, WI (US)

Correspondence Address: TUCKER ELLIS & WEST LLP, 1150 HUNTINGTON BUILDING, 925 EUCLID AVENUE, CLEVELAND, OH, 44115-1414, US

(21) Appl. No.: 11/748,735

(22) Filed: May 15, 2007

Related U.S. Application Data
(63) Continuation of application No. 10/722,997, filed on Nov. 26, 2003.
(60) Provisional application No. 60/429,849, filed on Nov. 27, 2002.

(51) Int. Cl. C12N 5/06

(52) U.S. Cl. 435/378; 435/381

(57) ABSTRACT

An apparatus 20 for the separation of a subpopulation of cells from an intact organ or other biological material is provided. The apparatus 20 includes: (1) a digestion chamber 24 that integrates the primary digestion process, (2) a measuring cylinder 26, (3) a cell collection chamber 28, (4) a heat exchanger 30 for raising and lowering temperatures in the digestion chamber 24 to activate or inactivate enzymes, (5) sensors 112, 114, 116, 118, 120, 122 to complete a closed feedback loop for allowing optimization of the digestion process, and (6) mock cells which mimic the cells to be harvested and which are used to fully optimize the process without unnecessary destruction of harvested cells. The manipulation of the digestion process may be manual or may be automated under computer control.

### CROSS-REFERENCE TO RELATED APPLICATIONS

This application claims priority to Provisional Patent Application Ser. No. 60/429,849 filed on Nov. 27, 2002, entitled CELL SEPARATION APPARTUS which is fully incorporated by reference herein.

### FIELD OF INVENTION

The present invention is directed generally towards a method and apparatus for separating and isolating cells from sample tissue, and more particularly, for controlling the separation of islet cells from pancreatic tissue for treatment of Diabetes Mellitus.

### BACKGROUND OF THE INVENTION

Diabetes is the fourth leading cause of death in the United States, resulting in one death every three minutes. Additionally, diabetes leads to many severe secondary health problems, such as amputations, and results in staggering overall financial costs to society. To date, there is no cure for diabetes.

In patients with Type 1 diabetes mellitus, insulin production by the pancreatic islets progressively declines and finally disappears, as the beta cells within the islets are destroyed by an autoimmune process resulting from an interplay between genetic and unknown environmental factors.

Currently, treatments for diabetes include one of three options: (1) insulin injections, (2) whole pancreas transplantation, or (3) islet cell transplantation. Insulin injections are at best trial and error estimations of levels of insulin to inject, resulting in the patient living at blood sugar levels which are out of balance with the body's needs. Insulin allows a diabetic to survive, but the effects of crudely controlled blood sugar levels lead to the many devastating consequences of the disease. When an excess of injected insulin drives blood sugar levels too low, the diabetic risks an immediate dramatic reaction that may include confusion, loss of consciousness, coma, and even death. When injected insulin is below the required amount, blood sugar levels rise, leading to damage to eyes, kidneys, nerves, heart, and blood vessels. Most diabetics are forced to operate at abnormally high blood sugar levels to avoid the more immediate and dramatic consequences of low blood sugar.

Whole pancreas transplantation suffers the problems of many transplantation procedures. First, transplanting a whole adult pancreas requires the use of immunosuppressive drugs to prevent organ rejection, and these drugs often have harmful side effects. Because of these hazards and the fact that whole pancreas transplantation is not a lifesaving procedure, it is usually performed only in people who also require a kidney transplant because of kidney failure, which is life threatening. Another pressing issue is the relative shortage of adult pancreases available. Even as whole pancreas transplantations are being performed on an increasing number of people, it is clear that there are not enough adult pancreases for everyone who might benefit from one. Further, whole pancreas transplant is a highly involved and invasive procedure with an extensive recovery period.

Islet transplantation, therefore, appears to be the most promising avenue for future development of a cure for diabetes. The pancreas includes two groups of cells: exocrine cells, which make up 95%-99% by weight or volume, and endocrine cells, which make up 1%-5% by weight or volume. The function of the exocrine cells is the manufacture of digestive enzymes that are not critical to health. The function of endocrine tissue is the manufacture of insulin, which is critical to glucose metabolism, and therefore life. The object of islet cell transplantation is to transplant live, viable islet cells and discard 99% of the exocrine pancreas, which is useless. Islets can maintain the body's insulin level in balance, and at the same time offer the possibility of being encapsulated in order to reduce or eliminate the immune response, thereby obviating the need for immunosuppressive medication.

Thus, islet cell therapies represent a promising alternative to the primarily used methods of treatment of diabetes because: (1) due to the small volume of cells to be transplanted, the procedure is potentially much less invasive than whole organ transplant, and the cells may be encapsulated which would obviate the need for immunosuppressive-suppressive therapies as is the case in whole organ transplant, and (2) the islets can function to auto-regulate the body's glucose levels which is not the case with insulin replacement therapies.

However, the number of donors from which viable islets may be harvested lags far behind the number of diabetes patients who would be acceptable candidates for such research. For example, there are sixteen million diabetics in the U.S. alone, with 2,200 new cases diagnosed every day, contrasted with less than 5,000 donors available each year. Thus, there is an obvious premium placed on insuring high quantity and quality yields of islet cells from each pancreas harvested.

Unfortunately, current methods of islet cell isolation are woefully insufficient in the qualities and quantities of yield. There are many various methods and devices which currently exist for separating component parts of a sample in order to obtain target cells. These methods include filters, centrifuges, chromatographs, and other well known separation methods. Other apparatus and methods exist for separating a particular cell subpopulation from a mixture of cells. These methods include chromatographic separation using columns, centrifuges, filters, separation by killing unwanted cells, separation by directly or indirectly binding cells to a ligand immobilized on a physical support, and separation using magnetic immunobeads.

In the prior art, various types of instruments for cell isolation have been proposed. For example, U.S. Pat. No. 5,079,160 discloses a method of obtaining purified, well-defined cells from intact organs. This method digests the distended organ with suitable proteolytic enzymes and allows for the harvest of the cell subpopulation by screening the effluent from the treatment of the organ with physiologically compatible medium. This harvest occurs by the use of a filtration screen which permits the passage of the desired cells, but prevents the passage of large particles.

U.S. Pat. No. 5,447,863 discloses a method and apparatus to concentrate and purify islets of Langerhans from a tissue suspension containing islets and tissue fragments. The tissue suspension is flowed through an inclined channel such that laminar flow is established. The islets settle toward the bottom and are drawn out.

U.S. Pat. No. 5,332,790 discloses a method of producing intact islets of Langerhans using a mixture of Hank's solution and 10% by volume fetal calf serum to ductilely distend the human pancreas. The exocrine tissue of the pancreas is digested at about 37° C. by an enzyme preparation of collagenase, trypsin, and proteolytic enzyme present in the mixture at a level of about 0.2% by weight.

U.S. Pat. No. 4,868,121 discloses a method of producing intact islets of Langerhans using a mixture of Hank's solution and 10% by volume fetal calf serum to ductilely distend the human pancreas. The exocrine tissue of the pancreas is digested at about 37° C. by an enzyme preparation of collagenase, trypsin, and proteolytic enzyme preset in the mixture at a level about 0.2% by weight. The digested pancreas is then comminuted, filtered and intact islets are recovered.

The method of pancreas digestion and islet cell isolation most commonly used today is a physical separation method that was first described in 1988. The general steps of this method are as follows: first, the donor pancreas is dissected of excess tissues, cannulated, and distended with a solution containing enzymes such as collagenase or liberase. Next, the islet cells are liberated from the exocrine tissues though the use of a continuous digestion. Pancreatic tissue is mechanically and enzymatically dissociated in a digestion chamber in the presence of a recirculating Hank's solution containing collagenase. This system consists of a lower stainless steel cylindrical chamber shaker containing the organ and several marbles. The solution is recirculated using a roller pump and temperature bath is employed in an effort to maintain the temperature of the fluid as close to 38° C. as possible to sustain optimum digestion. This digestion is performed manually. During the digestion, samples of islets are extracted, stained with diathizone, and examined under a microscope to gauge the extent of the digestion process. When it has been determined that the digestion is sufficiently complete (i.e., that islets have been sufficiently liberated from exocrine tissue), the flow is rerouted to a separate collecting flask where the enzymatic reactions are arrested by both diluting the islet containing solution and lowering its temperature to 4° C. Samples are then centrifuged to pellet the tissue, and the supernatant is drawn off and the tissue pellets are collected for purification.

The current method described above is, for the most part, performed manually in the lab, often requiring several lab technicians placed at several stations, each performing one step of the process. Problems have been noted in the current method of digestion/isolation particular to the manual method of digestion. Specifically, the manual method requires excessive manpower and labor, consumes a good deal of laboratory space, and perhaps most importantly to the goal of high purity yields, is not consistent on a day-to-day basis with regard to quality control. Thus, it would be desirable to provide an apparatus and method for islet cell separation which is automated and self-contained to reduce manpower and space requirements. It would be further desirable for such an apparatus and method to improve the quantity and quality of islet cells harvested from a pancreas.

### SUMMARY OF THE INVENTION

The present invention solves the problems and eliminates the drawbacks as described above in the background of the invention. It provides an integrated, automated process and apparatus for cell separation and isolation. In one aspect, this process may be automated. In another aspect, the present invention also provides materials which mimic the characteristics of the cell subpopulation to be harvested in order to facilitate the optimization of the cell separation process. In doing so, the present invention reduces manpower and space requirements, and increases the quality and thus the quantity of cell yield over that previously demonstrated.

More specifically, the apparatus of the present invention includes a number of constituent components. These include: (1) a digestion chamber that integrates the primary digestion process including, (2) a heat exchanger for raising and lowering temperatures in the digestion chamber to activate or inactivate the operative enzymes of the digestion process, (3) a temperature-controlled enzyme vessel for introducing enzymes to the digestion chamber, (4) sensors to complete a closed feedback loop to facilitate optimization of the digestion process, (5) a variable speed pump for causing flow of media and/or cells through a recirculation loop, (6) a sampling chamber within the recirculation loop which allows for sampling of the tissue/cells in media in order to monitor the progression of the digestion, (7) a cell collection chamber for holding isolated cells at the completion of the digestion process, (8) a network of tubing interconnecting the various components of the cell separation apparatus, and (9) a control for the flow of media and/or cells through the cell separation apparatus. Further, in one embodiment, the invention may include mock cells which mimic the cells to be harvested and which are used to facilitate optimization of the process without unnecessary destruction of the cells to be harvested.

All the physical components of the apparatus of the present invention may be in a single location, such as a fume hood. Additionally, the above-listed components may be located within or operatively connected to a control box, which may be used to facilitate monitoring and optimizing the digestion process. This reduces space requirements over previously described apparatus, which often included separate work stations. The consolidation of the apparatus also reduces manpower requirements. With the cell separation apparatus of the present invention, one lab technician may monitor the progression of the digestion, the optimization process, and harvesting of an isolated subpopulation of cells. Also, the cell separation process itself may be completely automated under computer control and monitored teleremotely.

The control of process parameters, such as temperature, may be achieved through the use of a central control system. In one embodiment, this control system may include a switchboard located on or operatively connected to the control box. In another embodiment, this control system may include a graphical user interface associated with a computer, which can be used to effect a particular variable at any point in the process. An operator may affect the parameters by using this control system. In yet another embodiment, the entire digestion process may be automated through computer control, thereby obviating the need for operator control through a control system. The control system may be operatively connected to low power consumption pinch valves which affect the temperature at any point in the process by rerouting the flow of hot and cold water to a particular stage of the process. In one embodiment of the present invention, the routing of hot and cold water to raise and lower temperature occurs through the use of a heat exchanger. Other regulated parameters may include pH, pressure, and dissolved oxygen concentration. The pinch valves may also be selected to determine the flow path for media and cells.

As mentioned above, the digestion process used in the present invention may be automated in order to reduce manpower requirements. One manner of such automation is to provide for computer control of the cell separation process. In one embodiment of the present invention, an operator can run and optimize an initial digestion by observing the progression of the digestion with mock cells. During this digestion, the various parameters, such as temperature, are monitored by the sensors and logged to the computer which operates as a data acquisition system. Subsequent digestions of actual organs may then be automatically controlled by the computer. In another embodiment of the present invention, even the initial optimization may be automated such that a digestion may be completely controlled by computer with the ability to optimize during the digestion process. During the digestion process, cells in the recirculation loop may automatically be diverted to a sampling chamber where the cells are digitally photographed and imaged. A computer may then compare the images of cells from the digestion chamber to imaged mock cells and thereafter automatically adjust the digestion parameters as needed in order to optimize and proceed with the digestion. In addition, the images used for comparison purposes may be provided by mock cells that are imaged concurrently with cells in the digestion process, or may be provided by archives of images of mock cells retained in the memory of the computer.

The control box of the cell separation apparatus of the present invention may act as an interface between the process of cell separation within the apparatus and the computer controlled data acquisition system. Among other purposes, the control box may provide a platform to control the entire operation of the cell separation. As described above, the process components required for the cell separation, including, but not limited to, the pump, the digestion chamber, the cell collection chamber, the heat exchanger, and the tubing may be operatively connected to the control box. A plurality of pinch valves, for controlling process flow in the various steps of the process, may also be mounted on or in the control box. These pinch valves may be solenoid-operated normally closed valves. An operator may operate the complete process by way of the control box. The control box may also house all control components for process indication, control and data acquisition. Temperature indicators for digestion chamber temperature, heat exchanger outlet temperature, and cell collection chamber temperature may be installed on the control box. The temperature sensors at these locations may be hooked up to these indicators through thermocouple connecting sockets. Indicators for pH, dissolved oxygen, and pressure may also be mounted on or in the control box. The pH sensor, dissolved oxygen sensor, and the pressure sensor may be mounted in the tubing of the cell separation apparatus. The control box further may house components of the computer and data acquisition process such as backplanes, interface boards, power supplies, and connecting boards.

Process indicators, including temperature, pressure, pH, and dissolved oxygen indicators, may have a retransmission current output facility. This retransmission output may be connected to analogue input modules on a first backplane of the data acquisition system. Additionally, analogue output modules, to control the speed of the pump and shaker oscillation frequency, may also be operatively connected to the first backplane. Digital output modules to control the operation of the pinch valves may be operatively connected to a second backplane. The first and second backplanes may be connected to analogue and digital I/O boards respectively. These I/O boards may generally be located inside a computer. The backplanes and the I/O boards may be connected to each other through a connection board.

The present invention also may include a software program which may include the graphical user interface to facilitate operator control of the various operations in the digestion process. The graphical user interface may use graphical indicators to show the parameters (such as temperature, pressure, pH, and dissolved oxygen) digitally and graphically against time. Process knobs may be used to control the pump speed and/or the shaker oscillation frequency. These parameters can be varied from 0 to 100%. The various steps in the digestion and cell separation process are selected by a main action switch. Alternatively, the software program may automatically manipulate process parameters as a result of comparisons of imaged cells of the digestion process to mock cells. The steps in the digestion process include: (1) filling of the digestion chamber and recirculating loop, (2) digestion of biological material, (3) emptying of the measuring cylinder, (4) dilution, (5) emptying of the recirculating loop, (6) sampling the results of the digestion, and (7) sampling the results of the dilution.

Additionally, the graphical user interface also may provide for supervisory control of the pinch valves to control a particular task. For example, the operator can either individually command pinch valve settings or can command a task, for example. In this latter case, the software of the graphical user interface automatically sets the required pinch valves to carry out the assigned task. During such an operation, the operator does not have to individually set each pinch valve. Additionally, the graphical user interface controls fail safe operation by determining if set limits for process parameters, such as pressure, are exceeded. If limits have been exceeded, the software automatically terminates pump, shakers, etc. Also, the graphical user interface may archive all data obtained during the isolation into a central database. This data may include all sensor measurements, all control actions, time stamps, and digital images. Other data that may be entered includes donor/recipient info, viability testing, etc., so that all relevant info on a given isolation may be located in a central place.

Additionally, the present invention provides a material which mimics the cell subpopulation to be harvested. This material may be a biological material, chemical composition, or other material used during the optimization process to calibrate the digestion and use as a standard against the actual cells of the subpopulation sought to be isolated during digestion. For example, this material may be in the form of mock islet cells used during optimization of a digestion process for islet cell separation from a pancreas. These mock islet cells may be beads that emulate many features of pancreatic islet cells. The beads are made of a material that approximates the density and dimensions of islet cells. The beads may have zinc ion attached to their surface which mimics the zinc that is released by islets as they make and release insulin. The beads can be visualized by the reaction between zinc ion and a chelating agent, such as dithizone. These chelating agents form a colored or flourescent complex with the zinc ion, either of which can be visualized with an appropriate microscope.

By the use of this apparatus, the present invention also provides a method whereby the preparation of clusters of cells with high yield and in relatively pure form can be achieved. This method is particularly useful for the production of preparation of islets, resulting in a harvest of a subpopulation of individual islets retained in native form. The method includes the digestion of the distended intact organ and perfusion of the organ with a carrier medium to remove islet cells. Yields of the islet cells are increased by the use of mock islets, described above, which allows for optimization of the method in the absence of the use of actual harvested islet cells. Recovery of the islet cells can then be followed by purification techniques such as size segregation. Additionally, the present invention provides for automation of the cell separation process.

Other features and advantages of the present invention will become apparent from the following detailed description, taken in conjunction with the accompanying drawings, which illustrate by way of example, the features of the invention.

### BRIEF DESCRIPTION OF THE DRAWINGS

FIG. 1 is a schematic depicting the apparatus used in the cell separation process of the present invention;

FIG. 1A is a schematic depicting the portion of the apparatus including the sampling chamber for optimizing the cell separation process of the present invention;

FIG. 2A is a schematic of the process steps of the cell separation process of the present invention;

FIG. 2B is a schematic of the process steps of the cell separation process of the present invention continued from FIG. 2A;

FIG. 3 is a schematic of the component layout of the control box of the cell separation apparatus of the present invention;

FIG. 4 is a schematic of the interior of the control box to depict the internal components of the control box;

FIG. 5A is a schematic of the sensors and wiring used to read and facilitate control of the cell separation apparatus of the present invention;

FIG. 5B is a schematic of the valves and wiring used to control the parameters of the cell separation apparatus of the present invention;

FIG. 6 is a schematic of the configuration of the hardware for computer control of the cell separation apparatus of the present invention;

FIG. 7 is a schematic of hot and cold water flow in the cell separation apparatus of the present invention; and

FIG. 8 is a schematic of the overall automated control of the cell separation apparatus of the present invention.

### DETAILED DESCRIPTION

With reference to the Figures, a cell separation apparatus 20 of the present invention includes a control box 22 which may house a digestion chamber 24. It may also include a measuring cylinder 26 and a cell collection chamber 28 interconnected with the digestion chamber 24. While in the illustrated embodiment, the digestion chamber 24 and measuring cylinder 26 are located within the control box 22 and the cell collection chamber 28 is located outside the control box 22, it will be recognized by those having skill in the art that any combination of components may be located within the control box 22. These components form a recirculating loop. The cell separation apparatus 20 may further include sensors 112,114, 116,118,120,122 which monitor parameters of the digestion process to complete a closed feedback loop for control and optimization of the digestion process. The cell separation apparatus 20 may further include a heat exchanger 30 for raising and lowering temperatures in the digestion chamber 24 and recirculating loop, a temperature controlled enzyme vessel (not shown), a variable speed pump 34, a shaker 36, and a central control associated with the control box 22 for manipulating digestion process parameters. Mock cells may be associated with the apparatus to aid in optimizing the digestion process.

As described briefly above in the summary of the invention, the procedure of isolation of a subpopulation of cells proceeds generally as follows. First, an intact organ in a physiologically compatible medium is distended at relatively low temperatures by the injection or infusion of an enzyme-containing medium which includes, but is not limited to, enzymes such as collagenase. A separate enzyme-containing medium may be used along with the physiologically compatible medium or, alternatively, the enzymes may be an ingredient of the physiologically-compatible medium. While in one embodiment the organ may be intact, those skilled in the art will recognize that the organ may be first dissected of excess tissues and cannulated, prior to being distended. Alternatively, the organ may be substantially dissected prior to being distended. The organ may be dissected in a dissection tray 214. One example of an organ to be used in the present invention is a pancreas. One example of cells to be separated in the present invention is islet cells. Those skilled in the art will recognize that other organs and cells may be used in the present invention. Second, following distention of the organ, the organ may be placed in the digestion chamber 24, which is a first chamber adapted to receive and organ or other biological material. Enzyme-containing medium is recirculated through the digestion chamber 24 containing the organ while raising the temperature of the medium in the digestion chamber 24 in order to activate the enzyme or enzymes. The digestion chamber 24 typically contains several Teflon marbles in addition to the organ and medium. The digestion chamber 24 is mounted within the shaker 36 and oscillated at controlled frequencies determined either manually by the operator or automatically by the computer as digestion occurs. The marbles provide agitation as the digestion chamber oscillates in the shaker 36. Other forms of agitation may be used. Third, the recirculation of organ, cells, medium, etc., through the recirculating loop may be monitored to detect the progression of the digestion and the separation of the desired subpopulation of the cells from the intact organ. Fourth, the process of separating the subpopulation of cells may be optimized by observation of or comparison of the cells being separated to mock cells which may be introduced into the digestion chamber 24. Alternatively, samples of cells from the digestion process may be collected from the cell separation apparatus 20 and compared against mock cells outside the cell separation apparatus 20. These mock cells may include material which mimics characteristics of cells of the desired subpopulation of cells that is to be isolated. Finally, the desired subpopulation of cells may be collected by terminating the recirculation of cells and medium through the recirculating loop and introducing fresh physiologically-compatible medium in an open system to circulate past and through the organ and into the cell collection chamber 28, which is a second chamber adapted to receive a subpopulation of cells. This may occur at a reduced temperature, so that any enzymes are rendered inactive.

More specifically, during the digestion process the organ may be maintained in a physiologically compatible medium and an enzyme-containing medium may then be introduced to the intact organ to cause the organ to be distended. Alternatively, an enzyme or enzymes may be added to the physiologically-compatible medium prior to applying the medium to an organ. The preparation of enzymes may include, but is not limited to, proteases, in order to catalyze the hydrolytic breakdown of proteins. Even more specifically, the proteases may include, but are not limited to, collagenase, which catalyzes the hydrolysis of collagen and gelatin. The medium does not necessarily need to include collagenase, but may include other proteases, such as liberase.

The intact organ used may, in one embodiment, be an organ in which general disruption of the tissue has not been affected by mechanical means. However, in alternate embodiments it may be necessary to divide the organ into smaller individual sections prior to introduction into the digestion chamber 24 in order to accommodate the size of the equipment and/or for convenience in handling. The organ may then be preserved at a low temperature (4° C.). However, the collagenase preparation may be injected at a higher temperature, in one embodiment in a range of about 24° C. to about 40° C. In one particular embodiment of the invention, the enzymes and/or enzyme-containing medium is introduced to the organ in the digestion chamber 24 at a temperature of about 38° C. The overall resulting temperature of the mixture is generally in the range of about 4° C. to about 28° C. In distending and digesting a whole intact pancreas in one embodiment of the process of the present invention, the pancreatic duct can be used as the passage to introduce the enzyme-containing medium to the interior of the organ. Other methods, such as direct injection, may also be used.

The enzyme-containing medium is chosen to be suited to the target organ, as will be understood by those skilled in the art. In a first embodiment, the enzyme-containing medium may include amounts of collagenase sufficient to digest a pancreas. For example, specialized collagenase preparations designed for hepatocyte isolation, pancreatic islet isolation, and adipocyte isolation are available commercially. In general, collagenase preparations may vary in the mixture of the specific enzymes they contain, and can be designed for the particular organ which serves as a substrate. For example, the collagenase-containing medium used in the first embodiment of the present invention may also include liberase.

The enzyme blend used during the digestion phase may, in one embodiment, be active at 37° C. and inactive at 4° C. In preparation for a cell separation, this enzyme blend may be reconstituted, brought to a predetermined concentration, and kept at 37° C. This occurs in the temperature controlled enzyme vessel. Once the isolation begins, the contents of the enzyme vessel are pumped in to distend the pancreas and the pancreas is then inserted into the digestion chamber 24. Any leftover enzyme may be poured into the system solution, either manually or by automation. It is this solution which flows through the cell separation apparatus 20, providing the medium for the digestion process.

Collagenase is commercially available, and is generally sold in various crude preparations of a number of proteases. The effective level needed for the invention disclosed herein depends on the nature of the collagenase preparation used and the cells to be separated, as will be recognized by those having skill in the relevant art. Such preparations may be available from Sigma, for example, which commercially provides a number of crude preparations which contain varying levels of proteases, such as trypsin, neutral and nonspecific protease, and others. In general, as used herein, collagenase is a term used to describe enzyme preparations which include collagenase and are effective in breaking down structural proteins. However, it will be apparent to those skilled in the art that other protenase preparations, even those lacking in collagenase, can also be used. In the first embodiment of the present invention, the collagenase-containing medium used is RPMI 1640 to which collagenase has been added. RPMI 1640 is commercially available from HyClone Laboratories, Inc.

The amount of enzyme used, as an effective amount, is one which is effective to digest the tissue of the target organ. As described above, in one embodiment of the present invention, this protease is collagenase and is present in concentrations which are capable of disrupting the relevant structural protein contained in the organ to an extent sufficient to free the desired subpopulation of cells from the organ. Since most structural protein comprises collagen, the use of a preparation containing collagenase is one general approach by which to obtain free cells. The concentration needed to be effective is variable, depending upon the organ and the preparation used. For example, for freeing islets from the pancreas, as in the first embodiment of the invention, a ratio of collagenase to medium in the range of about 0.5 ml/ml to about 3 ml/ml is generally effective. The effective concentration depends on the conditions of the digestion, including temperature, pH, and the extent of prior distension of the organ. Ascertainment of the amount of collagenase needed to be effective in a particular case will be well within the ordinary skill of the art, as the optimization of the digestion process will be provided by using the mock cells of the present invention, as will be discussed below.

As described above, the digestion of the organ and the separation and retention of the desired subpopulation of cells occurs within a physiologically compatible medium. Such a medium may be an aqueous buffer of appropriate ionic strength and pH to be compatible with living tissue. The medium may optionally contain supplements such as antibiotics or nutrients such as fetal bovine serum (FBS). Typical commercially available media of this type include Hank's solution, Ringer's solution, RPMI 1640, and the like. The pH and ionic strength conditions can be precisely adjusted in accordance with the organ, as will be apparent to those of ordinary skill in the art. These conditions can be monitored and adjusted throughout the digestion process by using a graphical user interface. In one embodiment of the present invention, as described above, RPMI 1640 is used as the physiologically compatible medium. In one embodiment, the pH in the apparatus is maintained in a range of about 6.8 to about 7.6.

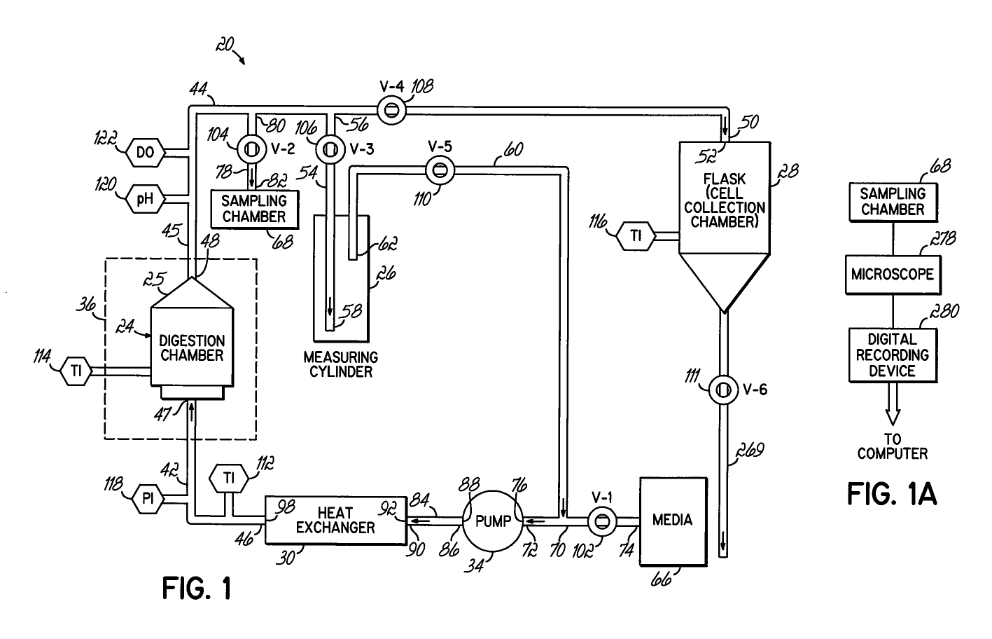

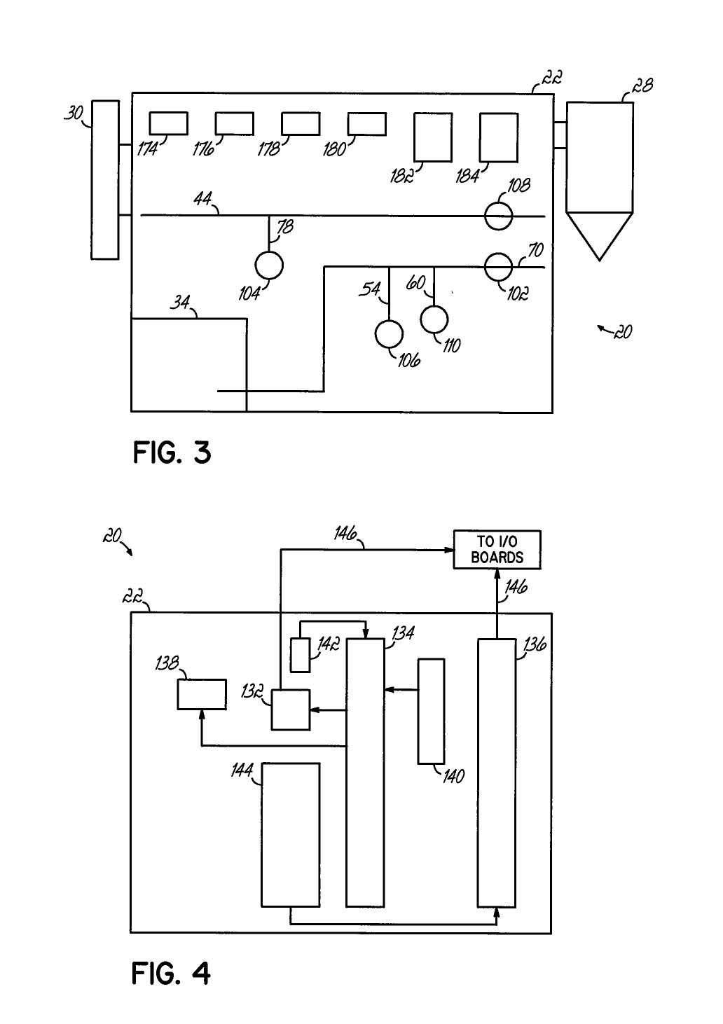

Turning now to the structure of the cell separation apparatus 20 of the present invention, the components may be located in a fume hood or other space such that they are self-contained within a single location in order to reduce manpower and space requirements. Referring now to FIG. 1, a schematic of the cell separation apparatus 20 and the components of the cell separation apparatus 20 of the present invention is shown. The apparatus 20, as described above, includes a digestion chamber 24 and a cell collection chamber 28. At least some of these may be located within the control box 22 (see FIG. 3) of the apparatus 20. However, it is not required that these components be located within the control box 22. An organ is distended within the digestion chamber 24 and isolated cells are ultimately collected in the cell collection chamber 28. The digestion process includes the use of other components of the cell separation apparatus 20. These include a temperature-controlled enzyme vessel (not shown) to retain enzymes such as collagenase or liberase; a temperature-controlling element, such as a heat exchanger 30, to raise and lower temperature at any point in the process to control the activation and inactivation of enzymes; a measuring cylinder 26 which is a third chamber in the fluid flow path that recirculates media and cells from the digestion chamber 24, through a recirculating loop, and back to the digestion chamber 24; tubes 42,44,54,60,70,78,84,94 to connect the various components; a central control associated with the control box 22 which may include sensors 112,114,116,118,120,122 to monitor parameters of the digestion process and pinch valves 102,104,106,108,110,111 to route the flow of media and/or cells through various components of the apparatus 20; and a variable speed pump 34 for pumping media and/or cells through the components of the cell separation apparatus 20. In one embodiment, each of these components may be located within the control box 22. Alternatively, only certain ones of these components may be disposed within the control box 22. The housing of the control box 22 also provides access to the components of the cell separation apparatus 20, such as by providing a moveable or removable panel, a door, or a lid, for example (panel, etc. not shown). This allows materials to be placed in and/or removed from various components of the apparatus 20. This includes placing the organ in the digestion chamber 24, placing media, such as RPMI 1640, in various containers, and removing cells or other material from the recirculating loop to monitor the progression of the digestion.

The digestion chamber 24 may be made of any material which is compatible with biological materials such that it does not interfere with the digestion process. During digestion, the digestion chamber 24 is mounted in the shaker 36. In one embodiment, the digestion chamber 24 may be made of a biocompatible polysulfone material, which is autoclavable and reusable. In one embodiment of the present invention, the chamber size is approximately 500 ml. However, the size of the digestion chamber 24 may range from about 250 ml to about 1000 ml. Alternatively, the size of digestion chamber 24 can be adjusted to meet the needs of the cell separation process, dependent on factors such as the organ to be digested, for example. A removable cover 25 may be attached to the top of the chamber 24, for example, by screw threads (not shown) and may be sealed by a gasket (not shown), such as by, for example, a conventional O-ring. A plurality of orifices may be disposed in the housing defining the digestion chamber 24. These orifices operate as ports to provide access to the interior of the digestion chamber 24 for sensors, for introducing media, or for removing media and/or separated cells. Additionally, a filter (not shown) may be disposed proximal to one or more of the orifices to filter any media passing from or to the digestion chamber 24. Attached to at least one of the orifices may be a first length of tubing 42. Such first length of tubing 42 provides transport for physiologically compatible media and enzyme-containing media to the digestion chamber 24. The tubing used in the apparatus 20 of the present invention may be, but is not limited to, silicone tubing. In one particular embodiment, the tubing used in the cell separation apparatus 20 of the present invention may be a Model No. L/S 16 (size 16) tube commercially available from Cole Parmer®. However, those of skill in the art will recognize that any material which is compatible with the media, cells, and/or mock cells, and does not interfere with the digestion process, may be used for the tubing of the cell separation apparatus 20 of the present invention.

The digestion and cell collection chambers 24,28 are connected one to another by a second length of tubing 44. A first end 45 of the second length of tubing 44 is connected to a first port 48 of the digestion chamber 24, and a second end 50 of the second length of tubing 44 is connected to a second port 52 located on of the cell collection chamber 28. A measuring cylinder 26 may be operatively connected as a component of the apparatus 20 along the flow path of the media, interposed between the digestion chamber 24 and cell collection chamber 28. As a result, the apparatus 20 provides at least two possible flow paths for the media: (1) from the digestion chamber 24, through the measuring cylinder 26, and back to the digestion chamber 24 in a recirculating loop, and (2) a path from the digestion chamber 24 to the cell collection chamber 28. To connect the measuring cylinder 26, the cell separation apparatus 20 includes a third length of tubing 54 having a first end 56 operatively connected to the second length of tubing 44, and having a second end 58 disposed within the measuring cylinder 26 in the illustrated embodiment. The measuring cylinder 26 forms part of the recirculating loop.

The measuring cylinder 26 serves a number of purposes. First, it functions as an opening in the system to prevent over-pressures. Without it the system would be totally closed to the atmosphere. Second, the dead space in the measuring cylinder 26 acts as an accumulator to modulate fluid flow and damp transients. Third, in one embodiment, the cylinder is made of glass so the effluent can be readily observed by the operator at a glance.

Fourth, the site of the measuring cylinder 26 can be used to insert various other sensor probes.

Although in the embodiment discussed above the measuring cylinder 26 is made of glass, the measuring cylinder 26 may be made of any material which is compatible with biological materials such that it does not interfere with the cell separation process. Such materials include, but are not limited to, polysulfone. In one embodiment of the present invention, the size of the measuring cylinder 26 is approximately 250 ml. However, the size of the cylinder 26 may range from about 100 ml to about 500 ml. Alternatively, the size of the measuring cylinder 26 may be varied to meet the needs of the cell separation process. A fourth length of tubing 60 may have a first end 62 dispersed within the measuring cylinder 26 to transport media, cells, and/or other material from the measuring cylinder 26. A second end 64 of this fourth length of tubing 60 may be operatively connected into a fifth length of tubing 70 which facilitates the transport of media, such as RPMI 1640, from a media container 66 to the digestion chamber 24. Thus, a recirculating loop is created from the digestion chamber 24, to the measuring cylinder 26, and back to the digestion chamber 24. As the media recirculates, samples of media containing cells may be periodically removed and observed in order to monitor and optimize the digestion. The samples may be removed from a sampling chamber 68. In the illustrated embodiment of the present invention, the sampling chamber 68 is operatively connected along the flow path of the media, interposed between the digestion chamber 24 and measuring cylinder 26. This sampling chamber 68 is a fourth chamber adapted to receive a portion of the subpopulation of cells. The sampling chamber 68 is connected along the recirculating loop by a sixth length of tubing 78 having a first end 80 operatively connected to the second length of tubing 44 and having a second end 82 operatively connected to the sampling chamber 68. The sampling chamber 68 is used to remove cells or other material from the recirculating loop in order to monitor the progression of the digestion. In the illustrated embodiments, a variable speed pump 34 and a heat exchanger 30 may be interposed along the recirculating loop between the media container 66 and the digestion chamber 24. This is described in greater detail below.

As described above, in general the digestion chamber 24 may also be connected along a flow path to the media container 66, so that media, such as RPMI 1640, may be transported from the media container 66 to the digestion chamber 24. As in the illustrated embodiment, the flow path may be interrupted by other components of the cell separation apparatus 20, such as a variable-speed, vacuum-pressure pump 34 and/or a heat exchanger 30. In this illustrated embodiment, an inlet port 76 of the pump 34 is connected to the fifth length of tubing 70 at a first end 72. A second end 74 of the fifth length of tubing 70 may be attached to the media container 66 holding the physiologically compatible medium. A heating circuit, such as may be provided by a heat exchanger 30, may be interposed along the flow path between the pump 34 and the digestion chamber 24. Thus, in the illustrated embodiment, a seventh length of tubing 84 may interconnect the pump 34 and the heat exchanger 30, and the first length of tubing 42 may interconnect the heat exchanger 30 and the digestion chamber 24. More specifically, a first end 86 of the seventh length of tubing 84 is operatively connected to an outlet port 88 of the pump 34 and a second end 90 of the seventh length of tubing 84 is operatively connected to an inlet port 92 of the heat exchanger 30. Likewise, a first end 46 of the first length of tubing 42 is operatively connected to an outlet port 98 of the heat exchanger 30 and a second end 47 of the first length of tubing 42 is operatively connected to the digestion chamber 24. The temperature provided by the heat exchanger 30 to the digestion chamber 24 and recirculating loop may be held at a constant temperature of about 37° C. in order to heat the physiologically compatible and enzyme-containing media to a temperature which allows for active digestion of the organ. However, the heat exchanger 30 may be alternatively operated to increase or decrease the temperature in the digestion chamber 24 and recirculating loop. A screening filter (not shown) may be placed in either or both of the digestion and cell collection chambers 24,28 to permit the collection of cells of a particular size, such as islet cells, and separate out other cell debris.

As described above, and referring to FIGS. 1 and 1A, the cell separation apparatus 20 includes a sampling chamber 68. This sampling chamber 68 may be used to remove cells as they progress through the digestion process, so that they may be observed and compared to mock cells to determine the progression of the digestion. This allows for the optimization of the digestion process by manipulating one or more of the process parameters following observation of the cells. In use, cells are periodically removed from the cell separation apparatus 20 via the sampling chamber 68. In one embodiment of the method of optimization of the present invention, these cells may then be stained and examined under a microscope to determine the progression of the digestion by comparing them to mock cells which have been stained. If the digestion is incomplete, one or more process parameters may be manipulated in order to enhance the quality of the digestion. If the digestion is complete, the recirculating loop may be closed off and the cells in the digestion chamber 24 may then be rerouted to the cell collection chamber 28. In determining the progression of digestion using actual cells of the cell subpopulation to be isolated, an operator would observe properties of the cells themselves and then observe markers or properties of the mock cells which mimic characteristics of cells of the actual subpopulation to be isolated. In an alternate embodiment, mock cells may progress through the digestion process with the actual cells of the cell subpopulation to be isolated.

In one embodiment of the present invention, the sampling of cells, analysis of the digestion process, and manipulation of one or more process parameters may be automated. In this embodiment, which will be discussed in greater detail below, after cells have been retrieved from the sampling chamber 68, they may be automatically stained and digitally imaged. These images may then be automatically compared to digital images of mock cells to gauge the extent of the digestion. The process parameters may then be automatically manipulated based on this automated comparison, or, if digestion is complete, the media and cells may be automatically routed to the cell collection chamber 28.

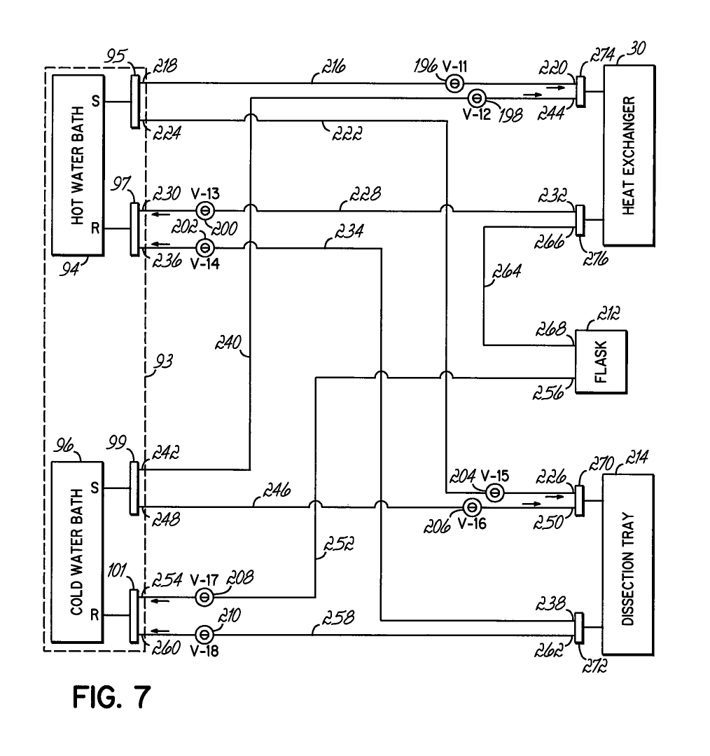

During optimization of the digestion process, an operator may wish to manipulate certain process parameters during the digestion, or, alternatively, certain process parameters may need to be automatically manipulated via computer control. In the cell separation apparatus 20 of the present invention, the manual manipulation of any parameter is provided for by a central control associated with the control box 22. In one embodiment, this may include a switchboard. In another embodiment, this central control may include the use of the graphical user interface running through the computer. This central control may allow for the manipulation of, for example, temperatures of the digestion and cell separation process at any point in the process by providing a plurality of pinch valves 196,198,200,208 (see FIG. 7) which can be used to reroute liquid flow to the heat exchanger 30 increase or decrease the temperature of the digestion at any point in the process. Thus, an operator may use these valves 196,198,200,208 to increase the temperature in the digestion chamber 24 if, upon observation and comparison of the cells with mock cells or stored cell/mock cell images, it is determined that the activity of the enzymes is not sufficient to successfully liberate cells from exocrine tissue. Other parameters which may be controlled include pH, pressure, and oxygen concentration. In one embodiment, the pH may be maintained in a range of about 6.8 to about 7.6. In one embodiment, the dissolved oxygen concentration may determined based on the dissolved oxygen concentration that is physiologically compatible for cells in biological materials which is well known to those having skill in the art. The dissolved oxygen concentration may be maintained at a range having a lower limit of 30 percent below a concentration that is physiologically compatible with cells of the subpopulation of cells to be isolated. The pressure to be maintained is based on the tubings and the connections used in the apparatus. In one embodiment, pressure may be maintained in a range from zero psi to an upper limit based on the pressure limit of the tubing and connection components used in the apparatus. In particular, the pressure may be maintained at a level that is below the upper pressure limit of the connections and tubings. Determining appropriate pressures by reference to pressure limits of components of apparatus is well known to those of skill in the art.

In the embodiment including a switchboard, switches (not shown), which may be used by an operator, are operatively connected to each pinch valve, so that by manipulating the switches, an operator can open and close any of the pinch valves 102,104,106,108,110,111,196,198,200,208,102, 204,206,208,210, thereby affecting a change in the desired process parameter or parameters or to reroute the flow of media and/or cells through the apparatus 20.

In one embodiment of the invention, the pinch valves used are low power consumption pinch valves, in order to handle the relatively low electrical loads of the apparatus of the present invention. In particular, the pinch valves may be Model No. 150P2NC24-06S, commercially available from BioChem. The pinch valves 102,104,106,108,110,111 for controlling the flow of media may be operatively connected to a passageway for fluid, such as the tubing of the cell separation apparatus 20, which is operatively connected to one or more components of the apparatus.

In one particular embodiment of the present invention, the pinch valves are solenoid-operated normally closed valves. However, those skilled in the art will recognize that any type of valve or pinch valves may be amenable to use in the apparatus 20 of the present invention. The pinch valves may include a hollow solenoid housing which contains a magnetizable solenoid bobbin and a solenoid coil. The solenoid housing is located on the lower portion of a valve body. The valve body may include a central cavity. The lower portion of a pressure block may be mounted in this central cavity. The upper end of the pressure block may bear on a section of a flexible length of tubing 44,54,60,70,78,216,222,228,234,240,246,252,258,264,269 of the apparatus 20. This flexible tubing may be mounted in a groove which extends diametrically across the valve body. The lower portion of the pressure block may be mounted on a circular disk made of a magnetic material. In normal use, the pressure block causes the portion of the flexible tube to collapse thereby preventing flow of fluid through the flexible tube. The pinch valve assembly is thus normally closed.

When the solenoid coil is energized via the leads, the disk, which is made of a magnetic material, is drawn away from the tubing and the force on the flexible tubing is released, causing the tubing to open and permitting flow through the tubing. The particular structure of the pinch valve, as described above, is not depicted in the Figures.

As described above, the pinch valves 102,104.106,108, 110,111,196,198, 200,202,204,206,208,210 may be operatively connected to the various lengths of tubings 44,54,60,70,78,216,222,228,234,240,246,252,258,264,269 and/or other components of the apparatus, such as the heat exchanger 30, in order to reroute the flow of media in the digestion process or affect various parameters of the digestion process, such as temperature. In the illustrated embodiment of the cell separation apparatus 20 of the present invention, and referring to FIG. 1, six pinch valves may be located in the following locations: (1) a first pinch valve 102 may be disposed along the fifth length of tubing 70 between the physiologically compatible medium container and the fourth length of tubing 60; (2) a second pinch valve 104 may be disposed along the sixth length of tubing 78; (3) a third pinch valve 106 may be disposed along the third length of tubing 54 in between the second length of tubing 44 and the measuring cylinder 26; (4) a fourth pinch valve 108 may be disposed along the second length of tubing 44 between the interconnection of the third length of tubing 54 and the cell collection chamber 28; (5) a fifth pinch valve 110 may be located along the fourth length of tubing 60 between the measuring cylinder 26 and the fifth length of tubing 70; and (6) a sixth pinch valve 111 may be located along the tube 269 for flow out of the cell collection chamber 28. Each of these pinch valves 102,104,106,108,110,111 may be opened and closed in order to route media, cells, and/or mock cells through the various tubing between the digestion chamber 24, and measuring cylinder 26 in order to optimize and complete the digestion process, and/or route the flow to the cell collection chamber 28 in order to separate and collect the desired subpopulation of cells.

Referring to FIGS. 1 and 1A, in one particular embodiment of the cell separation apparatus 20 of the present invention, the second pinch valve 104 may be used to obtain samples of the ongoing digestion phase. In particular, the second pinch valve 104 may be used to obtain samples, generally of approximately 1 ml each, of the system solution during the digestion phase of the isolation process. In one embodiment of the present invention, the cells to be isolated, and thus the samples obtained, are islet cells of a pancreas. The samples obtained are thereafter stained using a particular chemical that binds to the zinc which is present in insulin. Insulin is present in islet cells. In this way, islets in the solution can be distinguished from non-islet tissue. In one embodiment of the present invention, the samples may then be viewed manually under a microscope in order to determine the extent of the digestion. Alternatively, the samples may be digitally imaged and automatically analyzed by computer. Typically, three types of digested islets may be present in solution: (1) “embedded islets” are fully encased in pancreatic tissue and need more digestion in order to free them for harvesting; (2) “mantled islets” are partially encased in pancreatic tissue, but are not yet totally free; and (3) “free islets” are, as their name implies, fully digested and ready for harvest. As the digestion proceeds, the number of islets in category 1 diminishes, and those in categories 2 and 3 increase. After each sample has been analyzed, the contents may be discarded, as the stain may be toxic.

In one embodiment of the method of practicing the cell separation of the present invention, samples may be collected through the second pinch valve 104 into a small petri dish, which may then be transferred to a microscope for further examination by the human eye.

In an alternate embodiment of the present invention, the sampling mechanism may be automated, whereby the second pinch valve 104 may open to a sampling chamber, dye may be automatically injected onto the sample, and a recording device, such as a digital camera, may then record a picture of the cells. This digital camera may be operatively connected to a microscope. This picture may then be image processed to gauge the extent of digestion in an automated fashion by computer controlled comparison of the image of cells in solution to imaged data of mock cells. Depending on the information extracted from this image analysis, various parameters in the isolation system may then be automatically altered to control the digestion process. These parameters include, but are not limited to, temperature, pump speed, shaker speed, and solution concentration. Additionally, the image processing information may be used to determine a stopping point for the digestion phase of the isolation and then automatically transition the cell separation apparatus 20 into the dilution phase of the separation.

Other components of the cell separation apparatus 20 of the present invention, as mentioned above, may include a variable speed pump 34 and a heat exchanger 30. In the illustrated embodiment of the present invention, the variable speed pump 34 may be disposed between the fifth length of tubing 70 and the seventh length of tubing 84. When the apparatus 20 is set to recirculate media and cells through the recirculating loop, the pump 34 forces media from the physiologically compatible medium container 66 through the pump 34, the heat exchanger 30, and into the digestion chamber 24. From there the pump 34 forces the media to recirculate through the measuring cylinder 26, back through the pump 34 and into the digestion chamber 24 once again. Once completion of the digestion has been determined, the apparatus 20 may be set, either manually or automatically, to a dilution phase. In this phase, the pump 34 will force media and cells into the cell collection chamber 28. The pump 34 may be a variable speed pump 34 in order that media may be flowed through the digestion process at varying speeds, flow rates, and pressures. In a particular embodiment of the present invention, the variable speed pump 34 may be a Model No. U-07523 pump commercially available from Cole Parmer®. Once the cells have been collected in the cell collection chamber 28, they may be transferred to storage containers, such as flasks (not shown). To accomplish this, the sixth pinch valve 111 is opened, which allows media and cells to flow through tubing 269, and empty into a waiting storage container (not shown).

In the illustrated embodiment, the heat exchanger 30 may be disposed between the seventh length of tubing 84 and the first length of tubing 42. The heat exchanger 30 operates to transfer heat from one fluid to another, or alternatively, from a fluid to the environment. The basic heat exchanger 30 of the present invention consists of a length of pipe, a plurality of tubes disposed within the pipes, and first and second connectors disposed proximal to opposite ends of the pipe. According to the present invention, at least one of the plurality of tubes may be adapted to receive a first fluid. At least one of the plurality of tubes may be adapted to receive a second fluid. The plurality of tubes are in heat exchange relation to one another. The first fluid in one embodiment of the invention may be hot water or cold water. The second fluid, in one embodiment of the invention, may be media which may include cells and/or mock cells . The inlets and outlets may be operatively connected to the plurality of tubes. Thus, in the illustrated embodiment, an inlet port 92 of the heat exchanger 30 may be operatively connected to the seventh length of tubing 84 and an outlet port 93 of the heat exchanger 30 may be operatively connected to the first length of tubing 42. Thus, the media and cells may flow directly from the fifth length of tubing 70, through a first tube of the heat exchanger 30, and into the first length of tubing 42. This first tube of the heat exchanger 30 may be surrounded by a plurality of tubes. Thus, hot or cold water may be flowed through the plurality of tubes in order to respectively raise or lower the temperature of the media in the apparatus 20. In a particular embodiment of the present invention, the heat exchanger 30 may have a length of about 12 inches, and each of the plurality of tubes of the heat exchanger 30 has an outer diameter of about 5.2 mm and an inner diameter of about 5 mm. In this embodiment, the heat exchanger 30 may include 19 tubes arranged with 1 center tube, 6 tubes in a 0.64 inch diameter first circle encircling the center tube, and 12 tubes in a 1.20 inch diameter second circle encircling the first circle. The heat exchanger 30 additionally may include quick connect/disconnect functions operatively connected to the inlets and outlets, which allow them to be rapidly attached or disconnected from the cell separation apparatus 20.

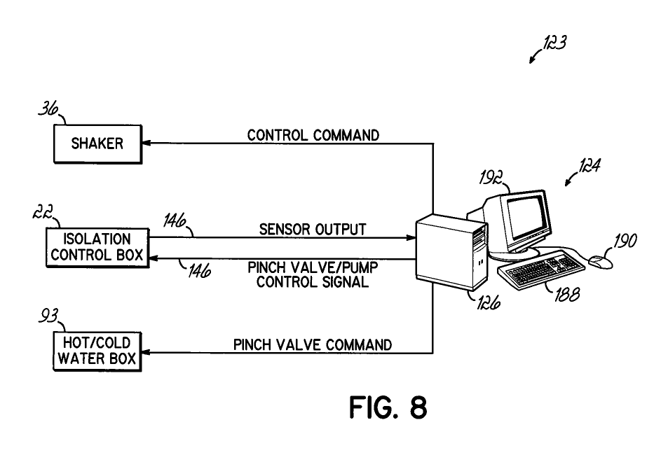

Referring now to FIGS. 1, 7, and 8 the source of water for heating and cooling by the use of the heat exchanger 30 may be provided by hot and cold water utilities box 93 which houses hot and cold water baths 94,96. Pinch valves inside this utility box 93 may be activated by the computer system 124 to direct hot or cold water, as needed, to the exchanger 30, depending upon which phase of the isolation process is running, and/or which parameters for temperature may have been altered. The utilities box 93 houses additional seventh, eighth, ninth, tenth, eleventh, twelfth, thirteenth, and fourteenth pinch valves 196,198,200,202,204,206,208,210 which are operatively connected to tubing within the utilities box 93 to supply hot and cold water to the islet isolation system. The heat exchanger 30 may also be operatively connected to a flask 212 and a dissection tray 214. As can be seen in FIGS. 7 and 8, the hot and cold water baths 94,96 of the utility box 93 may be operatively connected to the heat exchanger 30, flask 212 and dissection tray 214 via a plurality of tubes. In particular, an eighth length of tubing 216 is connected to a first end 218 to an outlet port 95 of the hot water bath 94, and at a second end 220 to a water inlet port 274 of the heat exchanger 30. A ninth length of tubing 222 is operatively connected at a first end 224 to an outlet port 95 of the hot water bath 94 and at a second end 226 to an inlet port 270 of the dissection tray 214. A tenth length of tubing 228 is operatively connected at a first end 230 to an inlet port 97 of the hot water bath 94 and at a second end 232 to a water outlet port 276 of the heat exchanger 30. An eleventh length of tubing 234 is operatively connected at a first end 236 to an inlet port 97 of the hot water bath 94 and at a second end 238 to an outlet port 272 of the dissection tray 214. A twelfth length of tubing 240 is operatively connected at a first end 242 to an outlet port 99 of the cold water bath 96 and at a second end 244 to the water inlet port 274 of the heat exchanger 30. A thirteenth length of tubing 246 is operatively connected at a first end 248 to an outlet port 99 of the cold water bath 96 and at a second end 250 to an inlet port 270 of the dissection tray 214. A fourteenth length of tubing 252 is operatively connected at a first end 254 to an inlet port 101 of the cold water bath 96 and at a second end 256 to the flask 212. A fifteenth length of tubing 258 is operatively connected at a first end 260 to the inlet port 101 of the cold water bath 96 and at a second end 262 to an outlet port 272 of the dissection tray 214. A sixteenth length of tubing 264 is operatively connected to a first end 266 to the water outlet port 276 of the heat exchanger 30 and at a second end 268 to the flask 212. In the illustrated embodiment, the seventh pinch valve 196 is operatively connected to the eighth length of tubing 216; the eighth pinch valve 198 is operatively connected to the twelfth length of tubing 240; the ninth pinch valve 200 is operatively connected to the tenth length of tubing 228; the tenth pinch valve 202 is operatively connected to the eleventh length of tubing 234; the eleventh pinch valve 204 is operatively connected to the ninth length of tubing 222; the twelfth pinch valve 206 is operatively connected to the thirteenth of tubing 246; the thirteenth pinch valve 208 is operatively connected to the fourteenth length of tubing 252; and the fourteenth pinch valve 210 is operatively connected to the fifteenth length of tubing 258. By opening and closing various ones of these pinch valves 196,198,200,202,204,206,208,210, an operator can reroute the flow of hot and cold water to the heat exchanger 30, flask 212, and dissection tray 214 in order to manipulate the fluid flow and, thus, the temperature of the digestion process of the cell separation apparatus 20.

The cell separation apparatus 20 of the present invention also includes a plurality of sensors 112,114,116,118,120,122 which are used to provide a closed feedback loop to allow for monitoring the progression of the digestion and cell separation process. The information obtained from this closed feedback loop thus aids an operator of the system in optimizing the digestion and cell separation process. Alternatively, the sensors 112,114,116, 118,120,122 may be used to create a data set which is used in automated control of the cell separation process. As information, such as temperature, pressure, pH, and dissolved oxygen concentration is received by the feedback loop through the sensors, the progression of the digestion can be monitored and the parameters of the process manipulated manually or automatically. In manual operation, once the parameters of a digestion have been determined, the parameters may be programmed into a central nervous system, such as may be provided by a computer system 124 to automatically control the cell separation activity of the apparatus 20. The sensors of the apparatus thus provide feedback to the control system. The closed feedback loop is a signal path which may include a forward path, a feedback path, and forms a closed circuit. In an alternate embodiment, computer control may be used to optimize and run the digestion even without the benefit of a previously logged and recorded data set.

As described briefly above, the data of the closed feedback loop of the present apparatus is provided by the plurality of sensors. These sensors may be used to monitor parameters of the cell separation process including, but not limited to, temperature, pH, pressure, and oxygen concentration. These parameters may be monitored at any point in the process merely by providing a sensor wherever monitoring such a variable is desired. The sensors may take readings of any variable constantly, or alternatively, at intervals ranging from about 2 seconds to about 15 seconds. The sensors may report this data back to the control system either constantly, or alternatively, at intervals ranging from about 2 seconds to about 15 seconds. As data is received, the operator or the computer system itself can determine any action to be taken in order to manipulate any particular variable at any particular point in the process.

As described above, a plurality of sensors 112,114,116,118, 120,122 may be provided in the cell separation apparatus 20 of the present invention. In one embodiment of the present invention, each of these sensors 112,114,116,118,120,122 may be disposed in a sensor port located in or on or in close proximity to the particular component or region of the process to be monitored. The sensors may then be operatively connected, such as by wire, to the computer controlled central nervous system of the apparatus. The present invention may also provide connection between each of the sensors and an associated display screen or indicator 174,176,178,180,182,184. These indicators 174,176,178,180,182,184 are disposed on the exterior of the housing and provide a readout of the current state of the process variable being monitored.

Referring now to FIGS. 1, 3, and 4, in the illustrated embodiment of the present invention, the cell separation apparatus 20 includes six sensors 112,114,116,118,120,122. These include three temperature sensors 112,114,116, one pressure sensor 118, one pH electrode 120, and one dissolved oxygen electrode 122. In one particular embodiment, the temperature sensors 112,114,116 may be Model No. TMQSS-125G-2.75″ sensors commercially available from Omega; the pressure sensor 118 may be Model No. PX177-050AI pressure sensor commercially available from Omega; the pH electrode 120 may be a Model No. U 05662-44 pH electrode commercially available from Cole Parmer®; and the dissolved oxygen electrode 122 may be a Model No. 53200-00 dissolved oxygen electrode commercially available from Cole Parmer®. The first, second, and third temperature sensors 112,114,116 record the temperature of the media at various points in the digestion process and provide this information to a display screen to be read by an operator. The temperature may be raised or lowered as desired to activate or inactivate enzymes in the enzyme-containing media. The manipulation of temperature may occur by use of the heat exchanger 30. This manipulation may be manual or automated.

In the illustrated embodiment of the invention, the sensors 112,114,116,118,120,122 are located as follows. The first temperature sensor 112 is interconnected into the first length of tubing 42 and monitors the temperature of the media after it has passed through the heat exchanger 30. The second temperature sensor 114 is interconnected with the digestion chamber 24 and monitors the temperature within the digestion chamber 24. The third temperature sensor 116 is operatively connected to the cell collection chamber 28 and monitors the temperature of the media within the cell collection chamber 28. The pressure sensor 118 is operatively connected to the first length of tubing 42 and is disposed between the first temperature sensor 112 and the digestion chamber 24. The pH electrode 120 is operatively connected to the second length of tubing 44. The dissolved oxygen electrode 122 is operatively connected to the second length of tubing 44 and is positioned downstream from the pH electrode 120. Each of these sensors 112,114,116,118,120,122 monitors a particular variable of the cell separation process and relays that information to a corresponding display screen or indicator 174,176,178,180,182,184. Additionally, the data collected by the sensors of the closed feedback loop may be relayed to a computer 126 in order to facilitate automated computer control of the cell separation process.

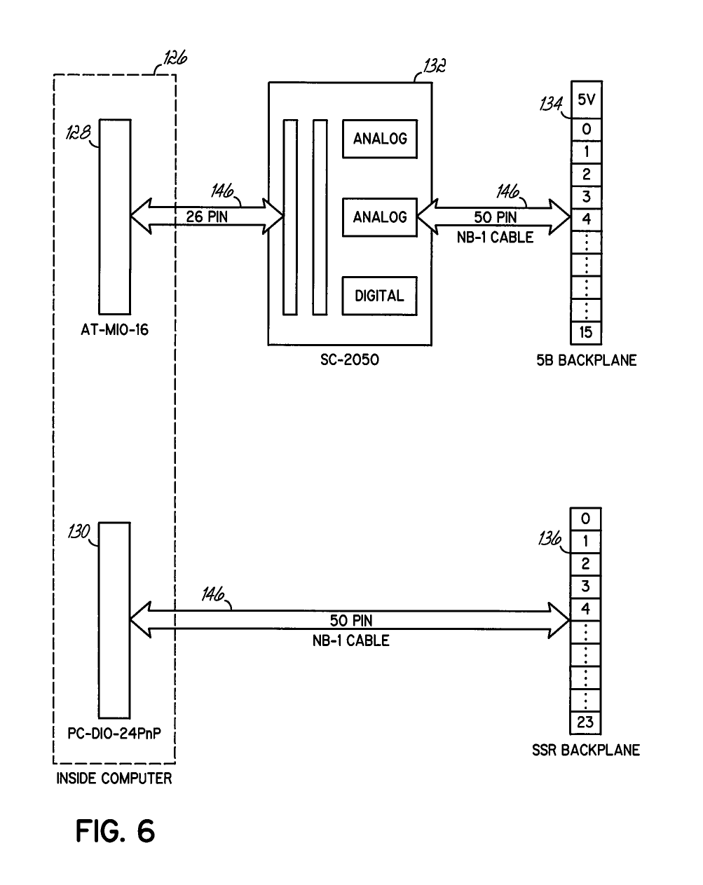

The cell separation apparatus 20 of the present invention, as described above, further may include automation provided by control system 123. In the illustrated embodiment, and referring now to FIG. 8, the components for this control system 123 include a computer system 124 having a computer 126 that is connected to the control box 22. Referring to FIG. 6, an analogue I/O board 128 and a digital I/O board 130 are mounted in the computer 126. Those boards are connected via cables 146 to the control box 22 that, as shown in FIG. 4, contains a connecting board 132, first and second backplanes 134,136, a shaker interface board 138, a distribution board 140, first and second power supplies 142,144 for the first and second backplanes 134,136, and cables 146 for interconnecting the various components to the I/O boards 128, 130 in the computer 126.

More specifically, the analogue I/O board 128 may be a Model No. AP MIO 16E 10 commercially available from National Instruments, and the digital I/O board 130 may be a Model No. PC DIO 24 PnP commercially available from National Instruments. The connecting board 132 may be a Model No. SC 2050 commercially available from National Instruments, which is used to connect both the analogue I/O board 128 and the digital I/O board 130 to the first and second backplanes 134,136. The first backplane 134 is a 5B 16 channel backplane, which may be Model No. 5B, commercially available from National Instruments. The second backplane 136 may be an SSR 24 channel backplane, which may be Model No. SSR, commercially available from National Instruments.

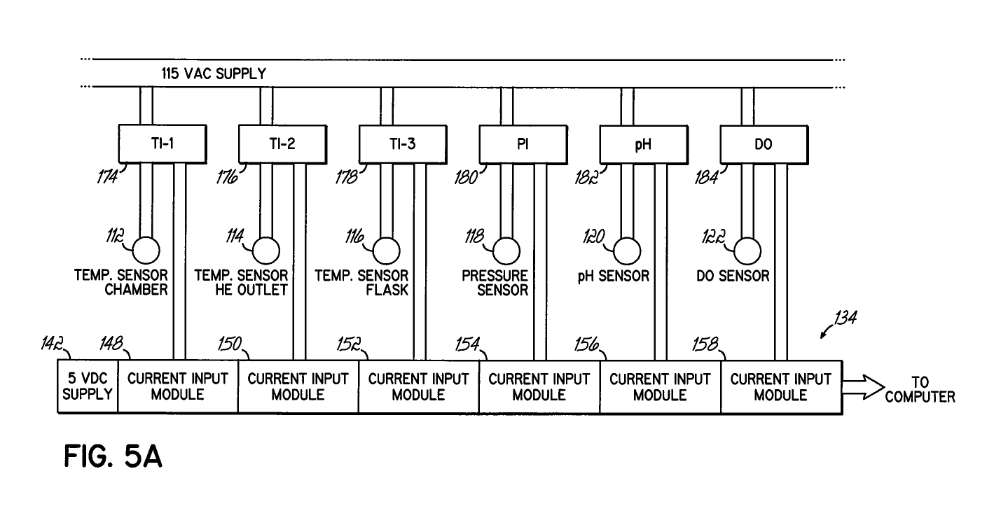

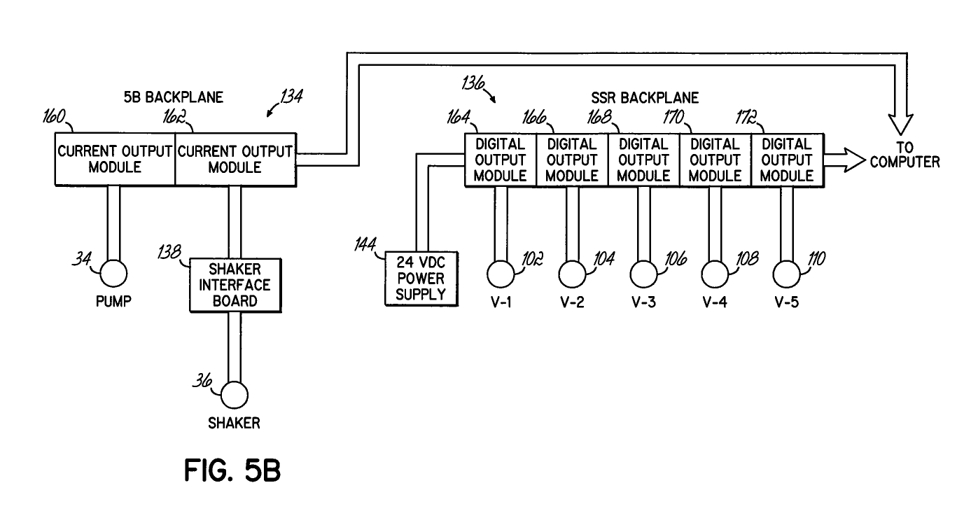

As shown in FIG. 5A, the first backplane 134 provides current input modules 148,150,152,154,156,158 connected to the various sensors and displays described above and, as shown in FIG. 5B, also includes output modules 160,162 connected to the variable speed pump 34 and the shaker 36. The output module 162 to the shaker 36 is also connected with the shaker interface board 138, which may be a Model No. KBSI 240D, commercially available from KB Electronics. The second backplane 136 connects digital output modules 164,166,168,170,172 with the first, second third, fourth and fifth pinch valves 102,104,106,108,110 of the cell separation apparatus 20, respectively. The analogue current input modules 148,150,152,154,156,158 may be Model No. 5B32-01 modules, commercially available from National Instruments. In the illustrated embodiment of the present invention, the cell separation apparatus 20 including computer control includes six analogue current input modules 148,150,152,154,156,158. Also, in the illustrated embodiment of the present invention, five digital output modules 164,166,168,170,172 may be Model No. SSR-ODC-5 modules, commercially available from National Instruments. The distribution board 140 may be a 115 VAC distribution board 140. The first power supply 142 may be a SVDC power supply for connection to the SB backplane 134. This first power supply 142 may be commercially available from Hughes Peters. The second power supply 144 may be a 24 VDC power supply for the second backplane 136, which may be a Model No. IHN24-3.6, commercially available from Hughes Peters.

The hardware components are connected to one another and to components of the cell separation apparatus 20 as follows. The second power supply 144 is connected to the second backplane 136, and the first power supply 142 is connected to the first backplane 134. The distribution board 140 is also routed into the first backplane 134. The second backplane 136 output is then be routed to the digital I/O board 130 in the computer system 124. The first backplane 134 output is routed through the connecting board 132 and into the analogue I/O board 128 in the computer system 124. One output module 162 of the first backplane 134 is connected to the shaker interface board 138 within the control box 22.

As described above, the second backplane 136 includes five digital output modules 164,166,168,170,172, connected to, respectively, the first 102, second 104, third 106, fourth 108, and fifth 110 pinch valves of the cell separation apparatus 20. The first backplane 134 includes six analogue current input modules 148,150,152,154,156,158 and two analog current output modules 160,162. Each of the current input modules 148,150,152,154,156,158 is connected to the sensors 112,114,115,118,120,122, respectively, and indicators 174,176,178,180,182,184, respectively, of the cell separation apparatus 20. For example, the first 148, second 150, and third 152 analog current input modules are operatively connected to the first, second, and third temperature sensors 112,114,116 and temperature indicators 174,176,178. The first temperature sensor 112 reads temperature in media as it flows from the heat exchanger 30 and routes that information to the first temperature indicator 174 which displays it to an operator. From the first temperature indicator 174, the information is routed into the first analogue current input module 148 and thereby is logged to the computer system 124. The second and third temperature sensors 114,116 read temperature in the digestion chamber 24 and cell collection chamber 28 respectively and that information is routed to the second and third indicators 176,178. From the second and third indicators 176,178 the information is routed to the second and third analogue current input modules 150,152 and is thereby logged to the computer system 124. The fourth analogue current input module 154 is operatively connected to the pressure sensor 118 and pressure indicator 180. The fifth analogue current input module 156 is connected operatively to the pH electrode 120 and pH indicator 182. The sixth analogue current input module 158 is operatively connected to the dissolved oxygen electrode 122 and dissolved oxygen indicator 184. From the pressure indicator 180, the information is routed into the fourth analog current input module 154 and thereby is logged to the computer system 124. From the pH electrode 120, information is routed to the pH indicator 182 and from there into the fifth analogue current input module 156. From the dissolved oxygen electrode 122, information is routed to the dissolved oxygen indicator 184 and from there into the sixth analogue current input module 158.

The first backplane 134 also may include first and second analogue current output modules 160,162 as described above. The first analogue current output module 160 is operatively connected to the variable speed pump 34 and the second analogue current output module 162 is operatively connected to the shaker interface board 138 and shaker 36.

In use, an operator monitors progression of the cell digestion and separation process by obtaining cells through the sampling chamber 68 of the measuring cylinder 26 and comparing characteristics of those cells to mock cells 40 which mimic similar characteristics. Based on the observations of the progression of the digestion, temperature may be increased or decreased, pressure or flow may be increased or decreased, etc. in order to increase or decrease the rate or length of the digestion process. For example, temperature may be raised or lowered by flowing hot or cold water respectively through the heat exchanger 30 while at the same time operating the pump 76 to flow media through the heat exchanger 30 to either raise or lower the temperature of the media. As this happens, the sensors for temperature 112,114,116, pressure 118, pH 120, and dissolved oxygen 122 constantly monitor the conditions of the digestion. Each of these sensors 112,114,116,118,120,122 as described above then relays this information to an indicator 174,176,178,180,182,184 on the control box 22 from which an operator can read and monitor the temperature, pressure, pH, and dissolved oxygen at any point at any time in the digestion process. The operator may then respond to the information on the indicators 174,176,178,180,182,184 by increasing or decreasing whichever parameter the operator so desires, based on comparison of cells to mock cells 40 pulled from the sampling chamber 68. At the same time, the sensors 112,114,116,118,120,122 relay this information to the indicators, the indicators in turn relay the information to analogue current input modules 148,150,152,154,156,158 on the first backplane 134 which log the information to the computer system 124. Thus, the computer system 124 continually records throughout the digestion process the set points for each of the parameters of the digestion process as relayed through the sensors and indicators.

The computer system 124 also logs information regarding the pinch valves 102,104,106,108,110, shaker 36, and variable speed pump 34. The computer system 124 logs when each of the valves is opened and closed during the digestion process at certain time points corresponding to the flow of media through the recirculating loop and cell separation apparatus 20. The computer system 124 also logs operation of the shaker 36 and pump 34 at various time points during the digestion process.

All the various data which is logged to the computer system 124 from the sensors 112,114,116,118,120,122, indicators 174,176,178,180,182,184, pinch valves 102,104,106,108,110, shaker 36 and pump 34 can then be recorded as a particular digestion program. For example, a first digestion program can be recorded to the computer system 124 for the digestion of pancreatic material for the separation of islet cells. A second program may be logged to the computer system 124 for the digestion of other organs for the separation of additional cells. Once a digestion process has been optimized and logged to the computer system 124, these programmed parameters can be used to automatically run subsequent digestions and cell separations in order to minimize manpower requirements and increase the quantity and quality of cell yield.

In one embodiment of the present invention, the data logged to the computer system 124 to set up programs can be controlled by a graphical user interface software program. In one embodiment, this graphical user interface may be based on a visual metaphor defining a monitor screen as a work space in which the contents of the controls are presented in window regions. The graphical user interface therefore may include a number of different of control objects, which enables the user to select from available options presented by the computer system's 124 operating system and/or application programs as well as by providing feedback to the user. Generally, the aspect of the present invention which is directed to the graphical user interface runs on a computer system 124.

In an alternate embodiment, as described briefly above, the digestion process of the cell separation apparatus 20 of the present invention may be automatically controlled via a computer system 124. As described above, the computer system 124 logs information from the sensors 112,114,116,118,120,122, indicators 174,176,178,180,182,184, pinch valves 102,104,106,108,110,196, 198,200,202,204,206,208,210, shaker 36 and pump 34. The computer system 124 may also contain stored in its memory digital images of cells, such as islet cells, having proceeded through digestion and having been stained. The computer system 124 may also contain stored in its memory digital images of mock cells. The computer system 124 may include a software program to recognize various characteristics of these mock cells as characteristics which are indicative of a completed digestion. The computer system 124 may open particular pinch valves to flow media and cells through the recirculating loop and may be programmed to periodically pull a sample from the sampling chamber 68. When this occurs, a digital recording device, such as a digital camera, operatively connected to the sampling chamber 68 will record an image of the cells in the digestion process after they have been stained within the sampling chamber 68. This digital camera is connected to the computer system 124 and logs the digital image recorded into the computer system 124 wherein it is compared to the images of cells stored in the memory of the computer system 124. The computer system 124 then may run a comparison of the various characteristics of these cells and of the archived images in order to make a determination as to whether or not a digestion is complete. If a digestion is not complete, the computer system 124 may then choose a variety of functions such as manipulating temperature, pressure, pH, etc., in order to facilitate the progression of the digestion. Once the computer system 124, as it continues to sample the digestion, “recognizes” that the cells of the digestion directly mimic those of the mock cells imaged in its memory, the computer system 124 may shut down the circulating loop by closing certain pinch valves and opening others to reroute flow of the media into the cell collection chamber 28. As this occurs, the computer system 124 may also instruct the cold water to flow from the cold water bath to the heat exchanger 30 in order to reduce the temperature within the cell collection chamber 28.

Referring to FIG. 8, an exemplary computer system, as described briefly above, includes a computer system 124 having a variety of external peripheral devices connected thereto. The computer system 124 includes a computer 126 and associated memory. This memory generally includes a main memory which contains the programs currently being executed on the computer 126 and which is typically implemented in the form of a random access memory (RAM). The associated memory also includes a non-volatile memory that can comprise a read-only memory (ROM), and a permanent storage device, such as a magnetic or optical disk, for storing all of the programs as well as data files. The computer 126 communicates with each of these forms of memory through an internal bus. The peripheral devices include a data entry device 188 such as a keyboard, and a pointing or cursor control device 190 such as a mouse, trackball, pen or the like. A display device 192, such as a cathode ray tube monitor or a liquid crystal display screen, provides a visual display of the information that is being processed within the computer 126. A hard copy of this information can be provided through a printer 194 or similar device. Also hooked into the computer 126 in the present invention may be other peripheral devices specific to the cell separation apparatus 20 including the sensors 112,114,116,118,120,122, pinch valves 102,104,106,108,110, indicators 174,176,178,180,182.184, variable speed pump 34, and shaker 36. Each of these external peripheral devices described above communicates with the computer 126 by means of one or more input/output ports on the computer 126.

In a computer system of this type, a graphical user interface, as described above, can be presented on the display device 192 through a software program to provide the user with a convenient mechanism to control the operation of the computer system 124 and to receive feedback regarding such operation. The control through this computer system 124 may be used to control the operation of the various components of the cell separation apparatus 20 in order to manipulate and optimize the digestion process. The graphical user interface forms part of the operating system of the computer 126 that is loaded from the permanent storage memory into the main memory when the computer system 124 is started, and which is executed while the computer system 124 is running. To provide input and output functionality, the graphical user interface includes various types of control objects which enable the user to select from available choices. Examples of such control devices include graphs, charts, and dials via which the user can monitor the status of the digestion, including various parameters such as temperature, pressure, pH, and oxygen concentration and may also interact with the graphical user interface in order to manipulate and change those various parameters. Typically, the user activates each of these various control objects by positioning a cursor on it, using the cursor control device 190, and actuating the object, by pushing a button or the like on the cursor control device 190. The computer system 124 then senses this operation and executes the function associated with the selected command.

In use, in one embodiment of the digestion process, the apparatus 20 is assembled after being sterilized and primed. The cell collection chamber 28 is filled with a physiologically compatible medium such as RPMI 1640. Additionally, the physiologically compatible medium container 66 and the digestion chamber 24 are filled with a physiologically compatible medium such as RPMI 1640. Positive pressure is exerted to drive media from the media container 66 into the digestion chamber 24.

An intact organ, such as a pancreas, is loaded into the digestion chamber 24 from the top and the top cover 25 is secured tightly. The variable speed pump 34 is started causing positive pressure to be exerted in the digestion chamber 24 and negative pressure to be exerted in the measuring cylinder 26. The third pinch valve 106 and fifth pinch valve 100 are open. This causes the media to circulate between the measuring cylinder 26 and digestion chamber 24 through the recirculating loop. At this point, the fourth pinch valve 108 is closed so that media does not circulate into the cell collection chamber 28.

Once the digestion chamber 24 is filled with media, the fluid will move from the digestion chamber 24 to the measuring cylinder 26 across the second length of tubing 44 and third length of tubing 54. A continuous recirculation of fluid is thus established which progresses from the digestion chamber 24, across the second and third lengths of tubing 44,54, through the measuring cylinder 26, across the fourth length of tubing 60, across the fifth length of tubing 70, through the variable speed pump 34, across the seventh length of tubing 84, through the heat exchanger 30, across the first length of tubing 42, and back into the digestion chamber 24. Enzymes from the enzyme vessel 32 are added to the media. As the collagenase distended pancreas in the digestion chamber 24 is digested, liberated cells flow through the second length of tubing 44 and third length of tubing 54 and enter the measuring cylinder 26. The progression of digestion is monitored by removal of cells through the sixth length of tubing 78 and sampling chamber 68, as described above, and comparing them to mock cells 40.

Once digestion is complete, the third pinch valve 106 may be closed to prevent the media and cells from continuing to circulate through the recirculating loop. Prior to the third pinch valve 106 being closed, the temperature of the media in the digestion chamber 24, measuring cylinder 26, and recirculating loop may be decreased to about 4° C. in order to inactivate the enzymes. At the same time, the fourth pinch valve 108 is opened in order to reroute the separated cells into the cell collection chamber 28.

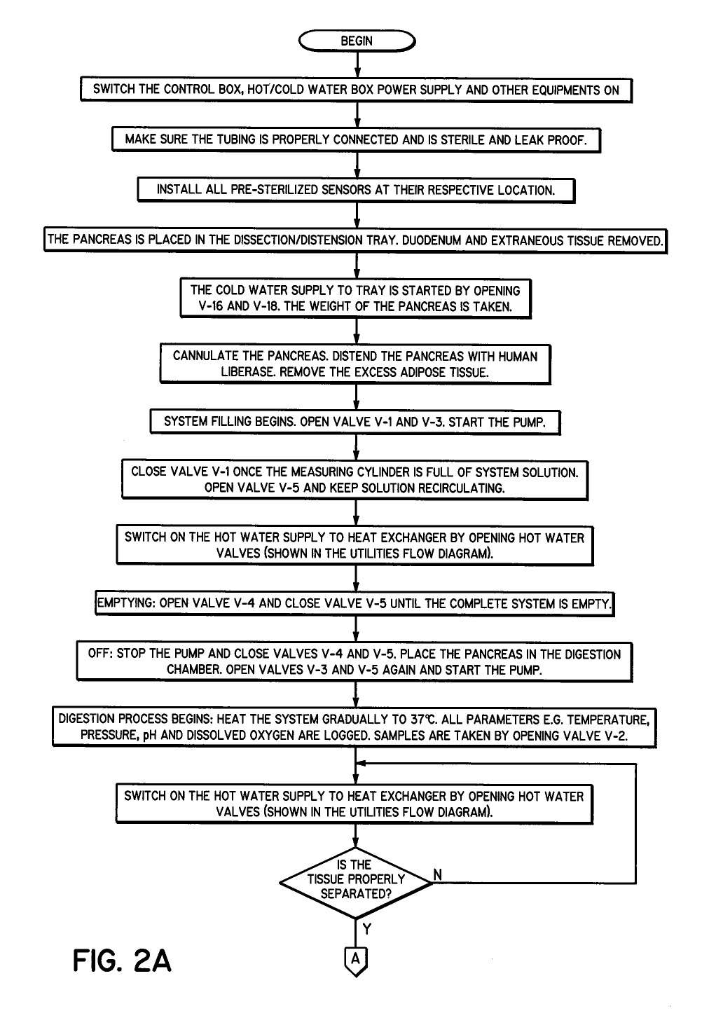

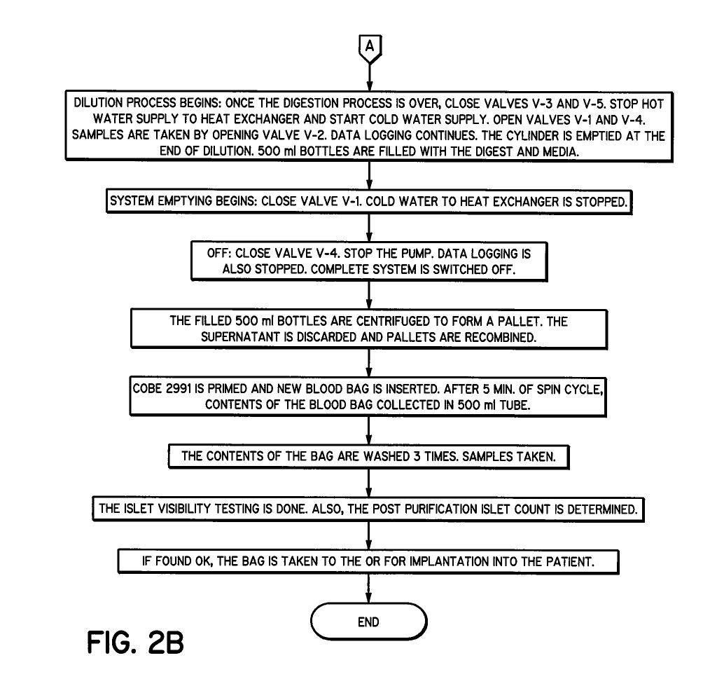

More specifically, and referring now to FIGS. 1-8, in the illustrated embodiment of the present invention, the digestion process is as follows. Initially, each of the first, second, third, fourth, fifth, and sixth pinch valves 102,104,106,108,110,111 are closed. An operator then switches the control box 22 on and makes sure that the interconnections with the computer system 124 are correct. The software program to run the digestion is then started. The software then opens the first pinch valve 102 and third pinch valve 106. This opens a passageway through the tubing of the cell separation apparatus 20 from the physiologically-compatible media container 66, across the fifth length of tubing 70, through the pump 34, seventh length of tubing 84, heat exchanger 30, first length of tubing 42, digestion chamber 24, second length of tubing 44, third length of tubing 54, and into the measuring cylinder 26. The pump 34 is then started by the computer 126 in order to begin the filling step of the cell separation process. This causes media to flow from the media container 66, through the digestion chamber 24, and ultimately to the measuring cylinder 26. The pump speed may be gradually increased. As the pump speed is increased, the digestion chamber 24 will start filling. Once the digestion chamber 24 is filled, the media level in the measuring cylinder will increase.

During this time, an organ to be digested, such as a pancreas, is being distended in preparation of undergoing digestion in the cell separation apparatus 20. This is done by placing the pancreas with media and enzymes, as described above, into the dissection tray 214. The eleventh pinch valve 204 and tenth pinch valve 202 are then opened. This causes hot water to circulate from the hot water bath 94, through the ninth length of tubing 222, through a portion of the dissection tray 214, through the eleventh length of tubing 234, and back to the hot water bath 94. This raises the temperature in the dissection tray 214, which activates enzymes to begin distension of the pancreas.

The rate of distention may be manipulated by raising and lowering the temperature in the dissection tray 214. Temperature may be lowered by rerouting cold water to the dissection tray 214 by closing the tenth and eleventh pinch valves 202,204 and opening the twelfth and fourteenth pinch valves 206,210. This shuts off the flow of hot water to the dissection tray 214 and routes cold water from the cold water bath 96 through the thirteenth length of tubing 246, to the dissection tray 214, through the fifteenth length of tubing 258 and back to the cold water bath 96. In one embodiment, the temperature of the water in the cold water bath 96 may be about 0.5° C.

Once the measuring cylinder 26 has been filled, the computer 126 instructs the first pinch valve 102 to be closed to prevent any additional media from entering the recirculating loop. Cooperatively, the fifth pinch valve 110 is opened. This prepares the system to begin the digestion process. With the pump 34 running, the media continuously recirculates through the loop. In one embodiment, the pump flow rate may be adjusted to about 90 ml/min. Next, the hot water supply to the heat exchanger 30 is switched on by the computer 126 in order to raise the temperature of the media passing through the heat exchanger 30. This is done by opening the seventh pinch valve 196 and the ninth pinch valve 200 which causes hot water to flow in a loop from the hot water bath 94, across the eighth length of tubing 216, into the heat exchanger 30, and from the heat exchanger 30, through the tenth length of tubing 202, and back into the hot water bath 94. In one embodiment of the present invention, the temperature of the water in the hot water bath 94 may be about 43□C. The pump 34 is then stopped and the third and fifth pinch valves 106,110 are closed. An organ to be digested, for example the now-distended pancreas, is placed in the digestion chamber 24. The third and fifth pinch valves 106,110 are then opened and the pump 34 started again. Thus the digestion step of the cell separation process may begin.

To begin the digestion, the temperature of the media in the recirculating loop is then gradually increased to about 37° C. in order to activate the enzymes. At this point, all parameters (i.e., temperature, pressure, pH, dissolved oxygen) are logged. This occurs by the first, second, and third temperature sensors 112,114,116, the pressure sensor 118, pH electrode 120, and dissolved oxygen electrode 122. Also, a sample of cells is taken. The samples are automatically taken by the computer by briefly opening the second pinch valve 104 which causes media containing cells to flow through the sixth length of tubing 78 and into the sampling chamber 68. Generally, the second pinch valve 104 is only opened long enough to allow about a 1 ml sample to flow into the sampling chamber 68 before the second pinch valve 104 is closed. In one embodiment, the computer 126 instructs samples to be taken every 3-4 minutes. This sample is routed in to a syringe (not shown) which is operatively connected to an outlet of the second pinch valve 104. From there the sample may be collected in a 35 mm diameter Petri dish where it is then stained. A microscope 278 may be proximal to the sample, such that the sample may be observed. A recording device, such as a digital camera 280 may be operatively connected to the microscope, When the second pinch valve 104 is opened to allow a sample to be taken, the digital camera 280 automatically records an image of the stained cells. This image is then transferred to the computer 126 and compared to imaged stained mock cells which mimic the islet cells harvested. The computer 126 determines whether the digestion is complete based on the proper separation of exocrine and endocrine tissue. If the digestion is not complete, the software program instructs the digestion to continue and may manipulate process parameters. The digestion and sampling continues until the compared images of the cells in the apparatus 20 are sufficiently “free” within a predetermined range as compared to the mock cells. This determination is made by use of the digital recording device, such as a digital camera, connected to the computer 126 running digital image processing software. The software acquires a digital snapshot of a sample taken from the sampling chamber 68, and processes it to obtain the various numbers and sizes of embedded, mantled, and free islets. The software then compares these values against empirically obtained thresholds. When the thresholds are satisfied, the computer issues a command to halt the digestion process, and begin the dilution through actuation of appropriate pinch valves. The software may even determine the rate of change of the numbers, sizes, and ratios of embedded, mantled, and free islets.

The image processing software can use either or both of comparisons to mock islet cells as well as comparisons to a database of archived islet snapshots, from previous isolations and/or taken under controlled experiments, in order to intelligently interpret images of samples pulled from the sampling chamber 68. The comparison undertaken by the software is a standard pattern recognition problem, and many algorithms well known to those of skill in the art exist to implement this task. Thus, the overall automated system replaces the human in the loop with an expert system.

Thus, there are at least three sources of information which could be used in determining the extent of digestion: (1) the expertise of the system operator, (2) an archive of digital images of cells that have been collected from previous isolations and/or taken under controlled experiments, and (3) the use of mock cells, such as mock islets.

Thus, in one embodiment, an operator may monitor the apparatus during an isolation. The operator may use his or her intuition about the digestion process to interpret views of digesting tissue under a microscope. The operator may be aided in this determination by the use of mock islets or by the use of archived digital images.

In an automated embodiment of the apparatus 20 of the present invention, the software of the computer (as described above) may involve standard pattern recognition which may be formulated on a rule base using the knowledge of the operator. This rule base forms the heart of a software based expert system that would control the apparatus in an automatic mode. This expert system may also include fuzzy decision making and/or trained neural nets tuned to mimic an operator's decision strategy. Such expert systems are well known to those having skill in the relevant art. For comparison purposes, as described above, the software may use the archive of digital images or images of mock cells taken concurrently with the digestion.

In one embodiment, the automation protocol may weight the real-time digital images obtained from an ongoing digestion against all three information sources described above (i.e., the expert system output, the archived images, and the mock islets) in order to track digestion and establish the best possible time at which to terminate digestion and begin dilution.

When the system is operating in manual mode (i.e., “human operator in the loop”), an operator is observing the digestion process. The operator can affect control of the digestion through the computer 126 via the graphical user interface as follows: 1) Temperature can be adjusted by actuating appropriate pinch valves and routing flows from the hot and cold water baths accordingly. By suitable cycling any temperature between 4° C. and 37° C. can be achieved and maintained. Secondary control can be achieved by adjusting the set-points of the water baths themselves; 2) Pressure is effected primarily by the speed of the pump 34. The measuring cylinder 26 also allows for some pressure relief and as an accumulation chamber to buffer flow transients. These two allow for correction of minor pressure variations from the desired pressure trajectory, which in one embodiment is basically a constant 0 pig. Significant over-pressures represent blockage of the filter in the digestion chamber 24. In order to prevent tubing and connections from failing, pump 34 and shaker 36 stoppage is required to maintain safe operation; 3) pH and dissolved oxygen concentration are monitored to ensure that they do not vary out of ranges necessary to maintain an solution environment suited for cell/tissue viability. These parameters can be adjusted thru the addition of buffer solution (RPMI, Hanks, etc.) to the effluent during digestion. In another embodiment, oxygenation may be added directly to the solution (e.g., via a tank, tube, bubble stone, and/or another pinch valve).

When the system is operating in automatic mode (i.e., closed loop thru the computer alone), the computer 126 can monitor these parameters through the sensor measurements. The computer 126 has control over pinch valves, pump speed, and shaker frequency. The computer 126 would compare measurements against desired trajectories and/or red lines and take appropriate action if the control objectives are not met via simple tracking and fail-safe operation modes built into the automatic operation software, as is well known to those having skill in the relevant art.

Once the digestion process is determined to be complete, a dilution step of the process begins. First, the third pinch valve 106 and fifth pinch valve 110 are closed. The measuring cylinder 26 is slowly emptied. The hot water supply to the heat exchanger 30 is halted by the computer 126 instructing the closing the seventh pinch valve 196 and ninth pinch valve 200 and the cold water supply to the heat exchanger 30 is started by the computer 126 instructing the opening the eighth pinch valve 198 and the thirteenth pinch valve 208 to allow water to flow in a loop from the cold water bath 96, through the twelfth length of tubing 240, through the heat exchanger 30, through the sixteenth length of tubing 264, through the flask 212, through the fourteenth length of tubing 252 and back to the cold water bath 96. This reduces the temperature of the media in order to inactivate the enzymes. In one embodiment, the temperature of the media is reduced to about 4° C. The first pinch valve 102 and fourth pinch valve 108 are opened in order to open the path to the cell collection chamber 28. During the entire process, the information from the sensors 112,114,116,118,120,122 has been logged by the computer 126. In one embodiment, the information from each of the sensors 112,114,116,118,120,122 is read and logged at intervals of 15 seconds. However, it will be recognized by those having skill in the art that the intervals of logging information can be set to any period desired by the operator.

The apparatus is then emptied by closing the first pinch valve 102. Cold water supply to the heat exchanger 30 is then shut off by closing the eighth pinch valve 198 and the thirteenth pinch valve 208. The action of the pump 34 then forces all media and isolated cells in the system into the cell collection chamber 28. In one embodiment, the speed of the pump 34 may be increased to 250-300 ml/min. Samples are taken periodically through the second pinch valve 104. Once no cells are observed, the fourth pinch valve 108 is then closed, and the data logging is stopped. The cells may be collected by opening the sixth pinch valve 111 which causes media and cells to flow through length of tubing 269 and to a container.

As described above, the steps of the isolation process are controlled from the graphical user interface on the computer 126. Also, the steps may be automatically controlled via the computer 126 by software to control the function of the components of the apparatus 20.

As described briefly above, the present invention also includes the use of mock cells in order to aid in the optimization of the cell separation process. These mock cells provide an internal control calibration standard for the automated system for cellular separation and isolation. The processing imaging, in turn, allows for process optimization and increased process reliability, minimizing human interaction. The measurement/monitoring of the process and archival of all relevant parameters involved during the isolation sampling and imaging, etc., will in turn, lead to increased speed, increased output, and decreased cost.

In one aspect of the present invention wherein the described subpopulation of cells includes islet cells, mock cells with the desired properties of islet cells will be used in the optimization process of the digestion. These mock cells may include a bead having a chelating agent, or ligand, covalently linked to the surface of the bead. Chelators may include, but are not limited to, EDTA (ethylenediaminetetraacetic acid), DTPA (diethylenetriaminepentaacetic acid), and ADA (aminodiacetic acid). Ligands coupled covalently to the bead via a tether permit the freedom of motion required for a zinc ion associated with the mock cell to be chelated. This complex is not colored and is stable at physiological pHs. The bead may then be visualized by introducing a stain, such as dithizone, thus forming a red-colored complex with free or partially ligated zinc.

In use, one embodiment of the present invention provides for beads as mock islet cells that simulate many features of pancreatic islet cells which may then be used to establish the optimal conditions necessary for the preparative separations of the cells, for example during centrifugation, thereby saving the very valuable islet cells themselves. The beads are made of a material that approximates the density and dimensions of islet cells, generally about 1.1 gm/ml density and 40 to 400 μm diameter. As described above, the beads have a zinc ion attached to their surface. The surface bound zinc mimics the zinc that is released by islet cells as they make and release insulin. The beads can be visualized by the reaction between the zinc ion and a chelating agent (such as dithizone or TSQ, etc.). These chelating agents form a colored or fluorescent complex with the zinc, either of which can be visualized with the appropriate microscope or can be automatically digitally imaged through the microscope, such as by a digital camera. These images may be logged to the computer 126 to be used in comparisons with cells to gauge the extent of the digestion process.

The present invention may, in one particular embodiment, include 50 to 200 micron diameter agarose beads with covalently attached IDA. Exposure of the beads to a solution of zinc results in binding of zinc to the bead surface. These beads are not colored or visualized by microscope. Adding dithizone causes the beads to turn red.

The mock islet cells of the present invention in one embodiment are added to the samplings of pancreatic tissue that are withdrawn or diverted from the digestion chamber 24 into the sampling chamber 68. In general, the mock cells, and in particular the mock islet cells, are not easily separated from the digestion mixture once added and so are not added to the pool of material which is ultimately to be implanted into a subject. Thus, in one embodiment of the present invention, the beads forming the mock cells are only added to samples prior to dithizone staining and analysis. As described above, the beads or mock islet cells are both physically and chemically much more resistant to degradative processes, such as those of the digestion process, than are real islet cells. In other words, any process that physically destroys the mock islet cells would first destroy the real islet cells. The chemical composition of the mock islet cells makes them completely resistant to any digestive effects of enzymes present in the pancreatic cell separation procedure. Thus, the status of the real islet cells with respect to the progress of the digestion may be judged separately using the unaffected mock islet cells as a calibration image.

The agarose beads used in a first embodiment of the mock islet cells of the present invention may more specifically be a spherical bead of about 6% agarose which has been cross-linked for chemical and physical stability and designated “fast flow” as will be appreciated by those having skill in the art. The treatment which gives the beads the capacity to hold or chelate divalent zinc ions is a chemical modification which introduces an iminodiacetate group. This property of metal bearing groups on beads makes them useful for metal chelate affinity chromatography, a wide-used technique known to those having skill in the art. The present invention involves specifically creating a zinc loaded bead, and then allowing the same zinc-chromophore (dithizone) interaction occur in the bead that happens when dithizone is used to stain the zinc within the real islet cells. In alternate embodiments of the present invention, almost any hydrogel that can be substantially modified with iminodiacetate groups might be used. Such hydrogels may include, but are not limited to, polymers of starch, dextran, agarose, alginate, agarose-dextrans, acrylamide, agarose-acrylamide, and others. The color reaction between the dithizone and zinc is not entirely specific to zinc, and other metal ions might give similar color reactions if these ions were loaded onto the beads in place of the zinc. Also possible is the substitution of the iminodiacetate group with some other metal chelating group to hold the zinc, or other metal ion, on the bead. The present invention also uses the proper affinity to balance zinc capacity and affinity. If the affinity is too low, the zinc will not be retained in the bead; if the affinity is too high, then it will not release the zinc to the dithizone in the proper conditions.

Additional properties relative to the zinc beads used as mock islet cells in the present invention are that, similar to the cells, they are partially translucent and therefore present their staining properties as a function of volume in depth and not just as a reflective or opaque surface. Additionally, the mock cells, and particularly the mock islet cells of the present invention, are not immediately toxic to pancreatic cells. Agarose is a moderately biocompatible polymer and, therefore, does not elicit any acute response from the actual islet cells. While it is anticipated that zinc ions may leach from the agarose beads and be taken up by actual islet cells, this only happens in a time frame of hours to days under conditions of a viable culture, but is not effective in creating such a problem in the few minutes of the actual analysis for optimization of the digestion process. This is because dithizone is considered a supravital stain in the sense that it is harmful or fatal to living islet cells in such that those cells having been treated are therefore not used for implantation.

While the invention has been disclosed by reference to the details of preferred embodiments of the invention, it is to be understood that the disclosure is intended in an illustrative rather than in a limiting sense, as it is contemplated that modifications will readily occur to those skilled in the art, within the spirit of the invention and the scope of the appended claims.

1-61. (canceled)

62. A method for isolating islets from a portion of a pancreas, comprising: introducing the portion of a pancreas to an islet processing solution that contains a digestive enzyme and that is characterized by a plurality of process control variables; circulating said islet processing solution around and through the portion of a pancreas and past a plurality of sensors, said plurality of sensors being exposed to said islet processing solution having an output that characterizes a state of one of the process control variables; controlling said plurality of process control variables of the islet processing solution during islet isolation with a process controller that is in communication with said plurality of sensors, said process controller having a process control interface and being capable of changing the state of said plurality of process control variables, wherein said plurality of process control variables include a temperature, a flowrate, a pH, a dissolved oxygen concentration, a dissolved nitric oxide concentration, an antibiotic concentration and an endotoxin concentration; separating the islets from the portion of pancreas; and collecting the separated islets.

63. The method of claim 62, wherein said controlling step comprises controlling said plurality of process control variables with a microprocessor temperature controller.

64. The method of claim 62, wherein said controlling step comprises controlling said plurality of process control variables with is a microprocessor controller.

65. The method of claim 62, wherein said controlling step comprises controlling said plurality of process control variables with a microprocessor computer.

66. The method of claim 62, wherein the temperature is adjusted by a recirculating fluid bath in thermal communication with the islet processing solution.

67. The method of claim 62, wherein the pH is controlled by a microprocessor pH controller between pH 6.00 and pH 8.00.

68. The method of claim 62, wherein the pH is controlled by a microprocessor controller between pH 6.00 and pH 8.00.

69. The method of claim 62, wherein the pH is controlled by a microprocessor computer between pH 6.00 and pH 8.00.

70. The method of claim 62, wherein the pancreas is a human pancreas.

71. The method of claim 62, wherein the pancreas is a transgenic porcine pancreas.

72. The method of claim 62, wherein the pancreas is a non-transgenic porcine pancreas.

73. The method of claim 62, wherein the pancreas is a transgenic mammalian pancreas.

74. The method of claim 62, wherein the pancreas is a non-transgenic mammalian pancreas.

75. A method for isolating islets from a pancreatic tissue, comprising:
a step for introducing the pancreatic tissue to an islet processing solution that contains a digestive enzyme and that is characterized by a plurality of process control variables;
a step for circulating said islet processing solution through the pancreatic tissue; a step for controlling said plurality of process control variables of the islet processing solution during islet isolation, the plurality of process control variables comprising: a temperature, a pH, a flowrate, a dissolved oxygen concentration, a dissolved nitric oxide concentration, a nitric oxide synthase activity, an endotoxin concentration, an endotoxin neutralizing protein concentration, an antibiotic concentration, an amino acid concentration, a dextran concentration, a heparin concentration, and a digestive enzyme activity;
a step for separating the islets from the pancreatic tissue while the process control variables are controlled; and
a step for collecting the separated islets.

76. The method claim 75, wherein the digestive enzyme activity is controlled by adding an antibiotic to the islet processing solution.

77. The method of claim 75 wherein:
said step for controlling one or more of said plurality of process control variables is accomplished with a process controller.

78. The method of claim 75 wherein:
one or more of said plurality of process control variables is controlled in said step for controlling.

79. The method of claim 75 wherein:
one or more of said plurality of process control variables is controlled with a process controller in said step for controlling.
